(19) United States
(12) Patent Application Publication
(10) Pub. No.: US 2017/0188554 A1
(43) Pub. Date: Jul. 6, 2017

(54) ANIMAL MODEL FOR STUDYING COMPLEX HUMAN DISEASES

(71) Applicant: UNIVERSITY OF SOUTH FLORIDA, TAMPA, FL (US)

(72) Inventors: JIA-WANG WANG, TAMPA, FL (US); RICHARD F. LOCKEY, TAMPA, FL (US)

(73) Assignee: UNIVERSITY OF SOUTH FLORIDA, TAMPA, FL (US)

(21) Appl. No.: 15/326,762

(22) PCT Filed: Apr. 18, 2016
(86) PCT No.: PCT/US2016/028148

§ 371 Date: Jan. 17, 2017

Related U.S. Application Data
(60) Provisional application No. 62/149,424, filed on Apr. 17, 2015.

(51) Int. Cl. A01K 67/027; G01N 33/50; G01N 33/58; C12N 5/0781

(52) U.S. Cl. CPC A01K 67/0278; C12N 5/0635; G01N 33/5052; G01N 33/582; A01K 2217/072; A01K 2217/206; A01K 2227/105; A01K 2227/106; A01K 2267/0387; A01K 2267/0393; A01K 2217/203; G01N 2500/10

(57) ABSTRACT

The invention concerns a non-human animal model useful for modeling complex human diseases; compositions comprising cell populations from the animal model having different genotypes for the same gene; methods for producing the animal model; and methods for studying a phenotype using an animal model or compositions of the invention.

### CROSS-REFERENCE TO RELATED APPLICATION

The present application claims the benefit of U.S. Provisional Application Ser. No. 62/149,424, filed Apr. 17, 2015, which is hereby incorporated by reference herein in its entirety, including any figures, tables, nucleic acid sequences, amino acid sequences, or drawings.

### BACKGROUND OF THE INVENTION

Mouse or other animal models are indispensable for studying the molecular mechanisms of human diseases and for pharmaceutical drug testing, as most of these studies cannot be done practically or ethically with humans. Most human diseases are complex diseases involving many genes and environmental inputs. The contribution of each gene to these diseases is usually small and is dependent on genetic background. To be reproducible, current mouse gene studies are dependent on establishing a congenic strain in which all mice have identical genetic material. The genetic variations can be eliminated. However, genetically, a congenic strain is analogous to a single human in the sense that one mouse strain/human cannot represent the entire population. To recapitulate the complex human disease, an existing line has to be backcrossed with many inbred strains, many times (10 generations or 2 years), to establish many congenic strains to mimic human genetic variations in the population. Practically, this is not possible due to cost and time, as it is estimated that at least 1001 mouse strains are required to represent the genetic variation in human population. Moreover, mice from a congenic strain still have genetic variations, and other variations (epigenetic and environmental) and experimental variations remain. Therefore, complex human diseases cannot be accurately modeled in mouse or other animals using the current techniques.

### BRIEF SUMMARY OF THE INVENTION

Common variable immunodeficiency (CVID) is the most common human primary immunodeficiency, which affects 1-2% of the population. The highly variable symptoms that are present in different CVID patients, including those with the same genetic causes, indicate complex interactions in multiple genes and environmental inputs, making it difficult to study this disease. Consequently, the etiology of over 80% of CVID cases is unknown. A small group of genes including the lipopolysaccharide-responsive vesicle trafficking, beach and anchor containing (LRBA) gene are found to cause CVID (Lopez-Herrera et al., 2012; Alangari et al., 2012; Burns et al., 2012). Unlike the other CVID genes, LRBA regulates the vesicle trafficking required for the regulation of one-third of human proteins (Wang et al., 2001). For example, LRBA regulates or may regulate many crucial proteins, e.g., NFκB, MAPKs, AKT, TNFα, IL10 and CVID receptors, and many components of the EGFR, NOTCH and RAS/MAPK pathways (Shamloula et al., 2002; Wang et al., 2004; Yatim et al., 2012). It is also involved in cell proliferation, apoptosis and autophagy (Lopez-Herrera et al., 2012; Wang et al., 2004). Furthermore, LRBA is a potential oncogene and is overexpressed in multiple cancers (Wang et al., 2004). For example, its overexpression is a molecular signature for breast cancer mortality and recurrence (Andres et al., 2013). These data demonstrate that LRBA has the features of central immune regulators like NFκB. Without being limited by theory, the inventors propose that LRBA controls multiple critical immune regulators and its deregulation causes immunodeficiency.

The inventors wished to study how LRBA regulates the critical immune regulators in vivo to better understand and treat immune disorders associated with deregulated LRBA. However, the lack of a good mouse model poses a critical barrier to test the inventors' hypothesis. Current mouse gene studies are dependent on establishing a congenic strain, which is analogous to a single human in the sense that one cannot represent the whole population. To recapitulate the complex human disease, an existing line has to be backcrossed with many strains, multiple times, to establish many congenic strains to increase genetic diversity to mimic human genetic variations in the population. This may not be feasible as it would cost too much money and time. Moreover, mice from such a congenic strain are still not genetically identical and it is demonstrated that the phenotypes originally attributed to the targeted gene may actually attribute to other genes (Eisener-Dorman et al., 2009; Ridgway, 2014). Therefore, it is not possible to accurately model complex human diseases in mice using the current techniques.

To alleviate this problem, the inventors have produced an all-in-one animal model (e.g., a mouse model) so that expression of a gene, such as Lrba, can be turned on/off and wild type (wt), heterozygote (het) and knockout (ko) cell populations can be produced and studied in a single animal and in a single tube (vessel) without the interference of variations. Phenotypes thus can be specifically attributed to the gene (e.g., Lrba) if they change in response to gene expression being switched on/off (FIG. 1). Lrba was selected as an example; however, any gene of interest can be utilized.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIGS. 1A and 1B. Strategy to study phenotypes in a single mouse. FIG. 1A: Lrba expression can be turned on\textcircled{2}/off\textcircled{3} by adding/withdrawing doxycycline (Dox). Tamoxifen (Tam) activates Cre resulting in the three genotypes\textcircled{4}. The removal of STOP between the two loxP (Triangles) will restore LRBA expression. The line colors of the cells (ovals) indicate the fluorescent proteins (FP) expressed: RFP (red, ko), CFP (cyan, wt) or both (red/cyan, het). X, no expression. FIG. 1B: Variation-free phenotyping technique in one mouse allows sensitive phenotyping\textcircled{2} and rapid genetic background change\textcircled{3} (blue to green) by inter-strain crossing (X). Phenotypes are shown as shapes. In the traditional model, the variations often mask the phenotypes (dash lines), resulting in ambiguous data\textcircled{1}.

FIGS. 2A-2G. Phenotype of Lrba KO depends on genetic background and LRBA regulates multiple critical immune genes. FIG. 2A: Germline transmission and backcrossing of Lrba knockdown (KD) allele. A 90% chimeric male founder (Lrba^{-/-}) produced by the Mouse Biology Program was mated with 129P2 (I) or C56BL/6J(II, 1) to obtain germline transmission. I.129P2 X 129P2 founder, II. C57BL/6J X 129P2 founder, III. 129P2 X B6;129P2-LrbaTm1Usf. 1. C57BL/6J X founder. 2-4. Backcrossing of 129P2-B6;LrbaTm1Usf with C57BL/6J. The triangle denotes the percentage of the C57BL/6J background increases. FIG. 2B: NFκB transcription activity was measured by an adenoviral NFκB luciferase reporter in Raji cells stably transfected with an LRBA shRNA KD plasmid and a control (WT) plasmid. FIG. 2C: Cytometric bead array assays of TNFα and IL-10 in Raji cells. The data are shown as a scatter plot with a bar graph of the mean and standard error of mean. FIG. 2D: LRBA KD protects cells from death induced by 500 nM of MG132 (proteasome inhibitors). FIG. 2E: Flow cytometry data show that LRBA KD downregulates CVID receptors in human Raji B cells. FIGS. 2F and 2G: LRBA knockdown deregulates MAPKs and AKT. Cell lysates from WT or LRBA KD stable Raji cells treated with or without LPS for the indicated time were analyzed by Western blot. Protein levels were quantified with Quantity One software. Phosphorylated (P-) protein levels were normalized to β-Actin or total AKT (ratio of phosphorylated/normalizing protein). FIG. 2F: Phospho-MAPKs (p38, JNK and ERK). FIG. 2G: Phospho-AKT. All experiments are representative of two to five separate experiments.

FIGS. 3A-3I. Construction of Lrba target vectors, Cas9/gRNA cleavage test and characterization of mouse embryonic stem (ES) cell clones targeted by CRISPR/HDR. The target vector is constructed from a 12.5 kb genomic fragment. The TCC splits the genomic fragment into 3.5 kb left and 9 kb right arms. FIG. 3A: Portions of sequencing trace show sequences around loxP-1 (FIG. 3A) (SEQ ID NO:3), loxP-2 (FIG. 3B) (SEQ ID NO:4), and loxP-3 (FIG. 3C) (SEQ ID NO:5). FIG. 3D: A secretable luciferase gene was inserted in place of Lrba through HR to functionally test the TCC, i.e. the tet-inducible system and Lrba promoters. The Luciferase assay was conducted in H293 cells transfected with the target vector with and without Dox. Without induction, the luciferase activity is negligible. FIG. 3E: SpeI digestion of PCR product using genomic DNA from ES cells transfected with Lrba sgRNA/Cas9 vector. SpeI digestion of the PCR product, 713 bp, produces 527 bp and 186 bp fragments in wt but cannot cut the CRISPR mutated allele (black arrow) as the SpeI site was destroyed by CRISPR. FIG. 3F: The Cas9/sgRNA can specifically digest the Lrba BAC subclone plasmid that contains the gRNA target sequence. The subclone plasmid has one PvuI site. FIG. 3G: PCR screening for Lrba knockin positive ES clones. The predicted size of the PCR product is 3.5 kb. FIG. 3H: The presence (+) of the third loxP site in these ES clones was detected by real time PCR using the loxP sequence as a Taqman probe (results shown at the bottom of FIG. 3G). FIG. 3I: Southern blot. EcoRV-digested genomic DNA was hybridized with a 5′ external probe with expected fragment sizes of 5 kb (wt) and 13 kb (targeted, T). The results agree with the PCR results and the Southern blot with Neo probe. 1. Linearized target vector (22 kb). Others: G418-positive clones.#4 and #9 incorrectly targeted. #6 Wt, Other clones were correctly targeted heterozygotes.

FIGS. 4A-4C. One-step generation of Lrba all-in-one mouse model by CRISPR technique. FIG. 4A: Generation of mouse model. (SEQ ID NO:6) The gRNA, Cas9 mRNA & target vector will be injected into a zygote. The gRNA (red sequence (nucleotides 6 to 25 of SEQ ID NO:6)) will guide the Cas9 nuclease to the target site and cleave the DNA. Homology directed repair (HDR) by the left and right arms (blue) of the target donor will then insert the TCC into the Lrba locus between the promoter and the Lrba translation start codon (ATG). Since the TCC contains a STOP cassette which will prevent the transcription of Lrba gene from upstream promoters, the insertion of the TCC will inactivate Lrba gene. E, EcoRV, S, SfiI. Red Blocks: Southern blot probes. FIG. 4B: Rescue of Lrba knockout. The excise of STOP by Cre recombinase will rescue the expression of Lrba and CFP. Inducible over-expression models: rtTA under Lrba promoter can bind to TRE to turn on the expression Lrba and CFP (dash arrows) in the presence of Dox; Reporter model: RFP and CFP reporter genes can be used to track Lrba promoter activity and expression. Lrba-p, Lrba promoter; Triangles: loxP, locus of cross P1 site; STOP, three different polyadenylation signal sites for transcription termination and polyadenylation of mRNA; rtTA, reverse tetracycline controlled transcriptional activator, TRE, tetracycline responsive element. X=No transcription. FIG. 4C: The impact of Lrba on the development of lymphocytes. Equal numbers of three genotypes of stem cells are produced by Tam-induced Cre recombination. The three genotypes are labeled with different colors: Wt, cyan; Het, cyan and red; Ko, red. Lrba deficiency results in more immature cells but less mature cells. The numbers shown are the ratio of the three cell types. The arrows between the genotypes indicate trans effects.

FIG. 5. Protocol for turning on/off Lrba expression. 20 μg/ml Dox in 2% sucrose will be supplied in the drinking water for αCreERT2 mice >6 wks of age for three days (red line) to activate Lrba transcription. The withdrawal of Dox will shut down the Lrba expression. Administration of Tam by i.p. will activate Cre (green line). Peripheral blood will be collected from the mouse before and after each treatment and subjected to flow cytometry.

FIGS. 6A-6C. Comparison of genotype-switching and labeling (GSL) technique with the traditional method. FIG. 6A: Traditionally, cells from each mouse (genotype) have to be analyzed separately, because if mixed, they cannot be distinguished. In contrast, with GSL, each genotype in a mixture is labeled with FP and can be distinguished. FIG. 6B: Consequently, e.g., to quantitate T cells of different genotypes, traditional method usually requires at least 12 mice and 12 tubes. While GSL requires only one mouse and one tube. FIG. 6C: More importantly, the phenotyping sensitivity can be greatly increased, e.g., one million times (2/0.000002) due to increased sample size and eliminated variations (for the convenience of calculation, a variation, e.g., 0.0001 was used). Wild type (AA), heterozygote (Aa) and knockout (aa). Calculations [90] were based on hypothetic averages and variations. *Total number required to obtain statistically significant data (p<0.05, power=80%).

FIGS. 7A-7C. Principle of Genebow and Cloning strategy. FIG. 7A: Cre recombinase-mediated deletion. loxP (locus of crossover in P1) consists of two 13 bp inverted repeats flanking an 8 bp core sequence that determines its polarity (denoted by dashed arrows). Cre cleavage produces two complementary protruding sequences: 5′-CATACA (blue), and 3′-TGTATG (orange). Sticky ends=complementary ends. Cre cleaves and ligates the two loxP sites in the same orientation, resulting in the deletion of the DNA between the two loxP sites and a circular DNA, which will be degraded and removed. FIG. 7B: Cre-mediated inversion. Cre cleaves the two inverted loxP sites. The cleaved intervening DNA is ligated back in an inverted orientation. As the sequences of the two loxP sites and the size of the intervening DNA remain unchanged, the reaction is reversible and the reaction rates of forward and reverse reactions is equal. In a balanced reaction, the numbers of the two end products, the original (i) and the inverted (ii), are equal. FIG. 7C: Genotype-switching and labeling (GSL). The exon of Lrba is flanked with two inverted fluorescent protein genes. The green FP† and Lrba are coexpressed. Cre-mediated inversion inactivates the co-expression but activates red FP expression. The wt allele is converted to ko allele and the green FP is replaced by red FP. Correspondingly, as each cell has two alleles, ko cells (with two ko alleles) will be labeled as red FP, wt cells as green FP, and heterozygous (het) cells as red/green FPs, and can be distinguished by flow cytometry. As stated above regarding FIG. 7A, the numbers of wt and ko alleles should be equal. This is important, as Cre recombination will not be a variable confounding the interpretations of results that are affected by genotype frequency. After the Cre reaction is balanced, Cre has to be removed. This can be easily done by stopping tamoxifen (TAM) treatment in the TAM-induced Cre system. Cre can be replaced with Dre, which, like Cre, is a highly efficient site-specific recombinase. When two recombinases are used, the targeted gene will be permanently inactivated like the current conditional gene knockout. †mNeongreen (NeonG) and mOrange2 have relative brightnesses over 35 [95]. EGFP and mRFP1 have a relative brightness of 36 and 13 have been successfully used to label cells [96]. NeonG is the brightest FP, three times brighter than EGFP. P=loxP, R=rox.

FIGS. 8A-8E. Characterization of Cre-mediated inversion by restriction enzyme digestion, targeting vector cloning and Time Line (FIG. 8E). The ApaI/BstI digestion will produce a 532 bp fragment when the intervening DNA is in original orientation (FIG. 8A), or a 2444 bp fragment when the intervening DNA is in inverted orientation (FIG. 8B). This assay will be used to analyze Cre-mediated inversion in bacteria detected by electrophoresis and in ES cells detected by Southern blot. FIG. 8C: Characterization of positive clone (pCAG-Invt) by restriction enzyme digestion (EcoRI/BglII). FIG. 8D: mNeonGreen expression in 293T cells transfected with the positive clone pCAG-Invt, indicating that the loxP and rox sites before the translation start codon ATG do not interfere with the expression of the downstream gene. The expression of Orange2 was not observed.

### BRIEF DESCRIPTION OF THE SEQUENCES

SEQ ID NO:1 is the nucleic acid sequence of the human wild-type Lrba gene (Homo sapiens LPS-responsive vesicle trafficking, beach and anchor containing (LRBA), transcript variant 1, mRNA. Accession number: NM_001199282:

SEQ ID NO:2 is the nucleic acid sequence of the mouse wild-type Lrba gene (Mus musculus LPS-responsive beige-like anchor (Lrba), transcript variant 1, mRNA. Accession number: NM_030695):

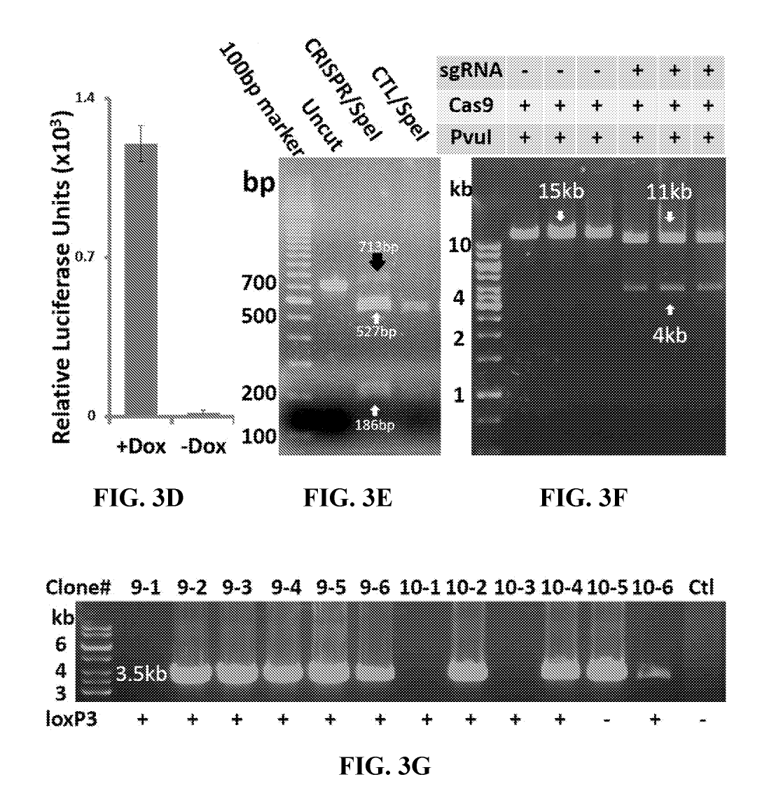

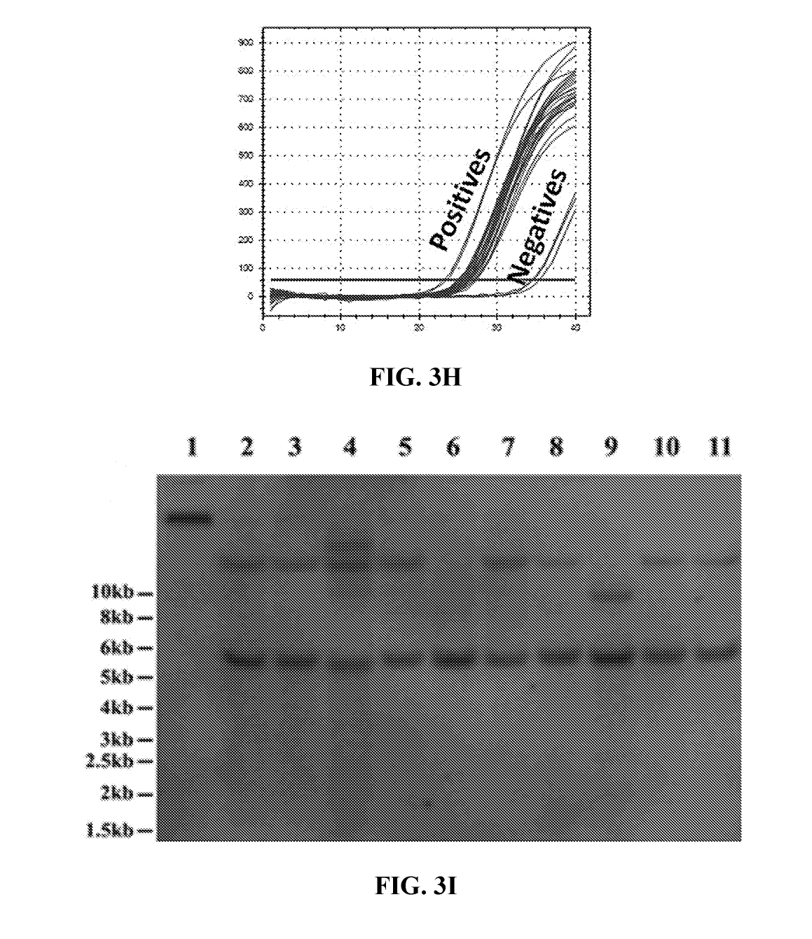

SEQ ID NO:3: Lrba/SD/loxP portion of Lrba BAC subclone plasmid (FIG. 3A).

SEQ ID NO:4: STOP/loxP portion of Lrba BAC subclone plasmid (FIG. 3B).

SEQ ID NO:5: P2A/Lrba portion of Lrba BAC subclone plasmid (FIG. 3C).

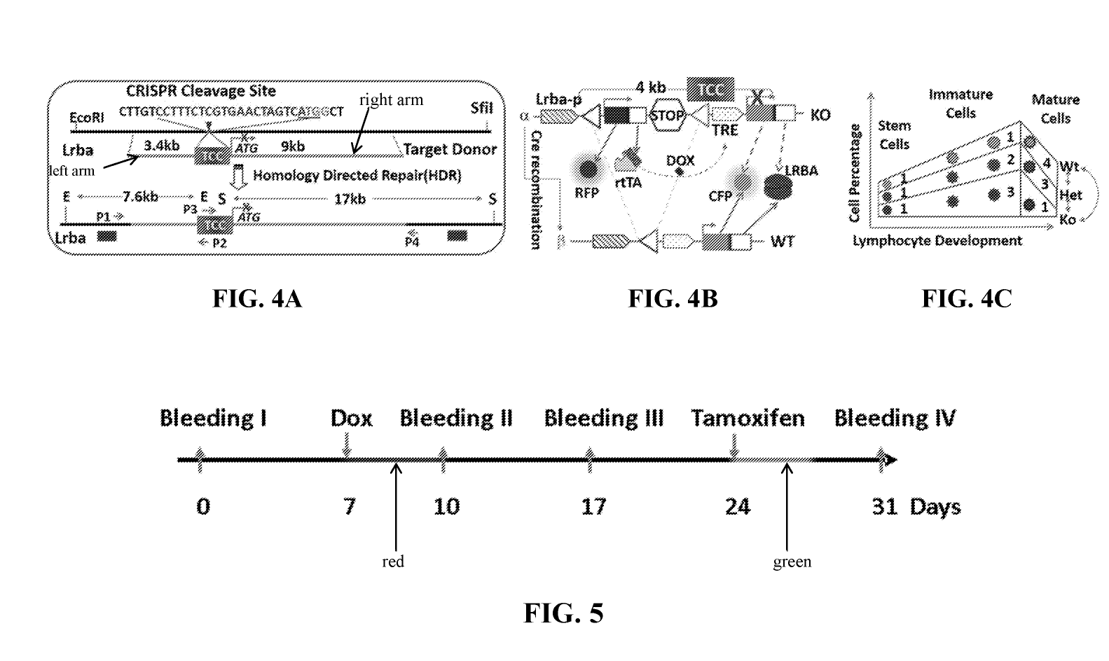

SEQ ID NO:6: transcription control cassette (FIG. 4A).

### DETAILED DESCRIPTION OF THE INVENTION

The invention concerns a non-human animal model comprising three genotypes for a single gene, resulting in three different cell populations for that cell type. Thus, each cell population has a different genotype for the gene. The invention concerns a non-human animal model comprising three populations of a cell type, wherein each cell population has a different genotype for a gene, wherein the three cell populations comprise:

(a) a first population of cells having a wild-type gene;

(b) a second population of cells heterozygous for the gene; and

(c) a third population of cells having an inactivated version of the gene;

wherein the three populations of cells include a transcription control cassette operably linked at the genomic locus of the gene.

Any gene of interest may be utilized to produce the non-human model. Lrba is used herein merely as an example. Various methods may be used for gene activation, such as insertion, deletion, substitution, and/or recombination.

The 2A peptide gene DNA sequence was used to link a flouresecnt gene and the two proteins to be co-expressed by the endogenous gene (Kim, Lee et al. 2011). Due to its small size (57 bp), high self-cleavability, and ability to produce an equal molar ratio of the two proteins, 2A is superior to IRES, which is large and causes differential expression of the two genes that it links (Kim, Lee et al. 2011). By placing the transcription control FP cassettes (TCFP) before the translation start codon (ATG), allows not only labeling of the endogenous protein but also tracking of the promoter activity of the endogenous gene. The STOP sequence in the casette is used to stop downstream gene expression. The Cre partial cleavage of the two loxP sites (triangle) will result in nine genotypes and cells with specific FP labeling.

The three populations of cells may comprise a first detectable label, a second detectable label, and a third detectable label, respectively, wherein each detectable label is distinguishable from the other detectable labels, wherein each detectable label is encoded by a nucleic acid sequence linked to the respective gene, and wherein the expression of the nucleic acid sequence is dependent upon expression of the gene (co-expressed) in the respective cell population. In some embodiments, the detectable label is a fluorescent label or luminescent label. In some embodiments, the detectable label is a fluorescent label selected from the group consisting of green fluorescent protein (GFP), red fluorescent protein (RFP), and cyano fluorescent protein (CFP).

The selected cell type will be that in which the three genotypes for that gene will exist. The cell type may be one in which the selected gene occurs naturally and is expressed, or is not expressed, or a cell type in which the gene is not normally present. Thus, the gene may be heterologous to the cell, and may be from the same species or a different species. In some embodiments, the three populations of cells are B cells.

Any gene may be utilized. In some embodiments, the three populations of cells are B cells, and the gene is lipopolysaccharide (LPS)-responsive beige-like anchor (Lrba) gene. Thus, the animal model may comprise three populations of B cells, wherein the three populations of B cells comprise:

(a) a first population of B cells having a wild-type lipopolysaccharide (LPS)-responsive beige-like anchor (Lrba) gene;

(b) a second population of B cells heterozygous for the Lrba gene; and

(c) a third population of B cells having an inactivated Lrba gene;

wherein the three populations of B cells include a transcription control cassette operably linked at the Lrba genomic locus.

The three populations of B cells may further comprise a first detectable label, a second detectable label, and a third detectable label, respectively, wherein each detectable label is distinguishable from the other detectable labels, wherein each detectable label is encoded by a nucleic acid sequence, and wherein the expression of the nucleic acid sequence is dependent upon expression of the Lrba gene in the respective B cell population. In some embodiments, the detectable label is a fluorescent label or luminescent label. In some embodiments, the detectable label is a fluorescent label selected from the group consisting of green fluorescent protein (GFP), red fluorescent protein (RFP), and cyano fluorescent protein (CFP).

In some embodiments, the animal model is a rodent, such as a mouse or rat, or a non-human primate such as a monkey or ape.

Another aspect of the invention concerns a composition comprising two or more of the populations of cells from the non-human animal model. Thus, the composition comprises a plurality of populations of cells from a single non-human animal, wherein said plurality of populations of cells comprise at least two of the following populations of cells:

(a) a first population of cells having a wild-type gene;

(b) a second population of cells heterozygous for the gene; and

(c) a third population of cells having an inactivated version of the gene;

wherein the three populations of cells include a transcription control cassette operably linked at the genomic locus.

Two or all three of the cell populations may be collected in the composition. In some embodiments, the populations of cells comprise a first detectable label, a second detectable label, and a third detectable label, respectively, wherein each detectable label is distinguishable from the other detectable labels, wherein each detectable label is encoded by a nucleic acid sequence, and wherein the expression of the nucleic acid sequence is dependent upon expression of the gene in the respective cell population. The populations of cells in the composition may be in isolated form, or may reside in a tissue sample collected from an animal model of the invention. The composition may be blood collected from the animal, such as peripheral blood.

In some embodiments, the cell populations are B cell populations.

In some embodiments, the cell populations are B cells and the gene is Lrba. In these embodiments, the composition comprises a plurality of populations of B cells from an animal model of the invention, wherein the plurality of populations of B cells comprise at least two of the following populations of B cells:

(a) a first population of B cells having a wild-type lipopolysaccharide (LPS)-responsive beige-like anchor (Lrba) gene;

(b) a second population of B cells heterozygous for the Lrba gene; and

(c) a third population of B cells having an inactivated Lrba gene;

wherein the three populations of B cells include a transcription control cassette operably linked at the Lrba genomic locus.

The animal models and compositions of the invention can be used for studying phenotypes. Thus, another aspect of the invention concerns a method for studying phenotypes (using the animal models or compositions of the invention), comprising:

providing a non-human animal model or composition of the invention; and

analyzing one or more of the phenotypes of the non-human animal model or composition in the presence and/or absence of an exogenous agent.

Phenotype analysis may involve analyzing the characteristics and/or behavior of one or more of the cell populations of the animal model or composition, which can be done, for example, by microscopy, flow cytometry, or other procedures known in the art.

Phenotype analysis may include measuring the detectable label of one or more of the cell populations of the animals or compositions and, optionally, comparing the measured detectable label to that of one or both of the other detectable labels.

The method may further comprise activing or deactivating the transcription control cassette to induce or inhibit expression of the gene, wherein a change in phenotype is indicative of a gene-dependent response (for example, in the case of Lrba, an Lrba-dependent response).

The exogenous agent may be a small molecule or biologic molecule that is administered to the animal model, or some other treatment administered to the animal.

### Animals

The non-human animal model is preferably a mammal. For example, the animal model may be a rodent or non-human primate. The single animal technique is especially useful to generate larger animal models that cannot be bred in large number (such as mice) which is normally required for statistical power using traditional methods.

In some embodiments, the animal is selected from the group consisting of a mouse, rat, guinea pig, hamster, gerbil, pig, cow, dog, wolf, coyote, jackal, and cat. In some embodiments, the animal model is a monkey or ape. In some embodiments, the animal model is a primate selected from the group consisting of a macaque, marmoset, tamarin, spider monkey, vervet monkey, squirrel monkey, and baboon. In some embodiments, the animal model is an ape selected from the group consisting of a gorilla, chimpanzee, orangutan, and gibbon. The animal model may be a hybrid of two non-human animals (e.g., dog-wolf).

Because the wild-type, heterozygous, and knockout B cells are present within the same animal, it is possible to accurately attribute changes in phenotype to Lrba as Lrba expression is switched on and off, without the need for control animals.

The animal model may have any desired genetic background. The animals may be crossed with many strains, and the gene (e.g., Lrba) may be studied with a wide genetic background, which is desired to recapitulate the complexity of human disease. As controls are not required, establishing congenic strains are not necessary. The all-in-one model of the invention can be crossed with many strains simultaneously and the function of a gene can be studied in a wide genetic background, which is desirable for recapitulating human complex diseases and can save time and resources.

The animal may be further modified at the genetic or epigenetic level so as to be useful in modeling a particular disease, such as cancer, cardiovascular disease, a metabolic disease such as diabetes, or a monogenic disease. For example, the animal model may be further modified to model Down Syndrome, cystic fibrosis, cancer, glaucoma, type-I diabetes, type-II diabetes, epilepsy, heart disease, muscular dystrophy, or gynecological tumors.

Down Syndrome—One of the most common genetic birth defects in humans, occurring once in every 800 to 1,000 live births, Down syndrome results from an extra copy of chromosome 21, an abnormality known as trisomy. The Ts65Dn mouse, developed at The Jackson Laboratory, mimics trisomy 21 and exhibits many of the behavioral, learning, and physiological defects associated with the syndrome in humans, including mental deficits, small size, obesity, hydrocephalus and thymic defects. This model represents the latest and best improvement of Down syndrome models to facilitate research into the human condition.

Cystic Fibrosis (CF)—The Cftr knockout mouse has helped advance research into cystic fibrosis, the most common fatal genetic disease in the United States today, occurring in approximately one of every 3,300 live births. Scientists now know that CF is caused by a small defect in the gene that manufactures CFTR, a protein that regulates the passage of salts and water in and out of cells. Studies with the Cftr knockout have shown that the disease results from a failure to clear certain bacteria from the lung, which leads to mucus retention and subsequent lung disease. These mice have become models for developing new approaches to correct the CF defect and cure the disease.

Cancer—The p53 knockout mouse has a disabled Trp53 tumor suppressor gene that makes it highly susceptible to various cancers, including lymphomas and osteosarcomas. The mouse has emerged as an important model for human Li-Fraumeni syndrome, a form of familial breast cancer.

Glaucoma—The DBA/2J mouse exhibits many of the symptoms that are often associated with human glaucoma, including elevated intraocular pressure. Glaucoma is a debilitating eye disease that is the second leading cause of blindness in the United States.

Type 1 Diabetes—This autoimmune disease, also known as Juvenile Diabetes, or Insulin Dependent Diabetes Mellitus (IDDM), accounts for up to 10 percent of diabetes cases. Non-obese Diabetic (NOD) mice are enabling researchers to identify IDDM susceptibility genes and disease mechanisms.

Type 2 Diabetes—A metabolic disorder also called Non-Insulin Dependent Diabetes Mellitus (NIDDM), this is the most common form of diabetes and occurs primarily after age 40. The leading mouse models for NIDDM and obesity research were all developed at The Jackson Laboratory: Cpefat, Lepob, Leprdb and tub.

Epilepsy—The “slow-wave epilepsy,” or swe, mouse is the only model to exhibit both of the two major forms of epilepsy: petit mal (absence) and grand mal (convulsive). It shows particular promise for research into absence seizures, which occur most often in children.

Heart Disease—Elevated blood cholesterol levels and plaque buildup in arteries within three months of birth (even on a low-fat diet) are characteristics of several experimental models for human atherosclerosis: the Apoe knockout mouse and C57BL/6J.

Muscular Dystrophy—The Dmd mdx mouse is a model for Duchenne Muscular Dystrophy, a rare neuromuscular disorder in young males that is inherited as an X-linked recessive trait and results in progressive muscle degeneration.

Ovarian Tumors—The SWR and SWXJ mouse models provide excellent research platforms for studying the genetic basis of ovarian granulosa cell tumors, a common and very serious form of malignant ovarian tumor in young girls and post-menopausal women.

### Reporters

Cell populations of the animal are preferably labeled with a detectable label (also referred to herein as a reporter) in order to detect and track gene promoter activity and gene expression, e.g., by flow cytometry. Thus, the reporter gene and the gene of interest are operably linked such that they are co-expressed. If a phenotype changes in response to gene expression being switched on or off, the phenotype can be specifically attributed to that gene.

Such detectable labels are known in the art, and include, for example, fluorescent reporter proteins encoded by fluorescent reporter genes. Preferably, each reporter can be detected in a living animal. Thus, the amount, distribution, proliferation, movement, properties, and behavior of the labeled cells can thus be assessed and, optionally, monitored.

Exemplary reporters include light-emitting reporters, such as fluorescent and luminescent reporters. Polypeptides that result in the generation of light in a living organism (bioluminescence) include, but are not limited to, various luciferases, green fluorescent protein (GFP), yellow fluorescent protein (YFP) and aequorin (Wilson and Hastings, Annu. Rev. Cell Dev. Biol., 1998, 14:197-230). Fluorescence reporters have many diverse uses, the most common of which are for fluorescence microscopy and also for flow cytometry. In both cases internal expression of the fluorescence reporter, using a reporter plasmid system, allows simple assessment of cell properties and/or behavior.

Luciferase is a luminescent molecule, and thus does not require excitation in order to generate light. It does typically require a substrate (e.g., luciferin, an aldehyde or coelenterazine), an energy source (e.g., ATP) and oxygen. In the case of bacterial luciferases, the genes encoding the substrate can be supplied the same vector as the gene(s) encoding the luciferase enzyme, thus eliminating the need for exogenously-supplied substrate (see, e.g., U.S. Pat. No. 5,650,135).

In some embodiments, the reporter for each B cell population is selected from the group consisting of red fluorescent protein (RFP), green fluorescence protein (GFP), yellow fluorescence protein (YFP), and cyano fluorescence protein (CFP). Polynucleotide cassettes encoding such polypeptides may be transfected into the target site as extra-chromosomal genetic elements (e.g., plasmids) or are stably incorporated into the genome (e.g., “hopped” in using, for example, a transposon).

If the reporter is a light-emitting reporter, method of measurement incluude using a photon detection device, such as an intensified CCD camera, a cooled CCD camera, or any other photon detection device with a high sensitivity. However, other methods may be used. For example, a light-emitting reporter may also be detected using a sensitive luminometer; a radioactive reporter may be detected by counts, X-ray imaging or scintillation.

The term “operatively linked” or “operably linked” refers to the connection of elements being a part of a functional unit such as a gene or an open reading frame (e.g., encoding LRBA). Accordingly, by operatively linking a promoter to a nucleic acid sequence encoding a gene product such as a polypeptide the two elements becomes part of the functional unit—a gene. The linking of the expression control sequence (promoter) to the nucleic acid sequence enables the transcription of the nucleic acid sequence directed by the promoter. By operatively linking two heterologous nucleic acid sequences encoding a polypeptide the sequences becomes part of the functional unit—an open reading frame encoding a protein or proteins comprising the amino acid sequences encoded by the heterologous nucleic acid sequences. By operatively linking two coding sequences, the sequences can be co-expressed.

LRBA mutation causes CVID, which is highly heterogeneous, genetically, immunologically and clinically. It is associated with many diseases and conditions including infections, chronic lung disease, autoimmunity, hepatitis, granulomatous disease and cancers (Lopez-Herrera et al., 2012; Alangari et al., 2012; Burns et al., 2012; Gathmann et al., 2012; Eibel et al., 2010; Park et al., 2008). The association with autoimmunity is paradoxical but intriguing as it provides a unique opportunity to study the etiology of autoimmunity associated with many chronic human diseases (Podjasek and Abraham, 2012). The CVID symptoms are highly variable among patients including those with the same genetic causes. For example, the same LRBA mutation presented in two nuclear families of cousins causes hypogammaglobulinemia in one family but not in the other (Alangari et al., 2012), indicating complex interactions in multiple genes and environmental inputs. This makes it difficult to study this disease. Unlike other CVID genes, which include cell membrane receptors [CD19, CD20, CD21, CD81, inducible costimulator (ICOS), B cell-activating factor receptor (BAFFR), transmembrane activator and calcium modulator and cyclophilin ligand interactor (TACI)], LRBA causes more severe and potentially life-threatening CVID and a higher incidence of autoimmune problems (Lopez-Herrera et al., 2012; Alangari et al., 2012; Burns et al., 2012; Wang and Lockey, 2014). It regulates vesicle trafficking (Wang et al., 2001; Cullinane et al., 2013), which is essential for the proper function of about one-third of human proteins (Kholodenko, 2003; Wang et al., 2010; Wiley, 2003). Thus, LRBA potentially regulates many proteins, e.g., the LRBA orthologue, rugose, interacts with the multiple components of the EGFR, NOTCH and RAS/MAPK pathways (Shamloula et al., 2002; Wang et al., 2004; Yatim et al., 2012), and the preliminary data herein show that LRBA regulates NFκB, MAPKs, AKT, TNFα, IL10 and CVID receptors. It is also an oncogene candidate as it is overexpressed in breast, lung, colon and kidney cancers, and its promoter activity is significantly increased by oncogene E2F1 and inhibited by tumor suppressor p53 (Wang et al., 2004). For example, LRBA knockdown inhibits cancer cell growth (Wang et al., 2004), while LRBA overexpression is a molecular signature for breast cancer mortality and recurrence (Andres et al., 2013). Moreover, it is involved in cell proliferation, apoptosis and autophagy (Lopez-Herrera et al., 2012; Wang et al., 2004). These data demonstrate that LRBA has the features of central immune regulators such as NFκB. The inventors propose that LRBA regulates critical immune regulators and its deregulation (null, down-regulation and over-expression) causes immunodeficiency.

Current mouse models cannot recapitulate most human diseases including LRBA-caused CVID as these diseases have highly variable phenotypes. Traditional mouse studies are dependent on establishing a congenic strain, which is analogous to a single human in the sense that one cannot represent the entire population (Vogel, 2003). To recapitulate such a complex human disease, an existing line has to be backcrossed with many other strains about 10 times (2 years) to establish many congenic strains to increase genetic diversity to mimic human genetic variations in the population. This is not possible due to cost and time. Moreover, mice from a congenic strain still have genetic variations due to the tightly-linked genes flanking the targeted gene and single nucleotide polymorphisms (Eisener-Dorman et al., 2009; Ridgway, 2014). Therefore, it is not implausible that the phenotypes originally attributed to the targeted gene actually be attributable to other genes (Eisener-Dorman et al., 2009; Ridgway, 2014). To alleviate this problem, the inventors propose the production of an all-in-one animal model (e.g., an all-in-one mouse model) in which Lrba expression can be turned on/off in a spatiotemporal and trackable manner, so that, for example, wt, het and ko B cells with respectively distinct reporter signals (e.g., different FP colors) can be produced in a single animal. Thus, phenotypes can be studied by flow cytometry in a single animal and in a single tube with high resolution (sensitivity) as there are no interferences from the genetic, epigenetic, environmental and experimental variations, and can be specifically attributed to Lrba if they change in response to Lrba expression being switched on/off. This animal model will allow the inventors to test this hypothesis, confirming the in vitro data that LRBA regulates many critical immune regulators, NFκB, MAPKs, AKT, TNFα, IL10 and CVID receptors to demonstrate that like NFκB, LRBA is one of most important immune regulators.

The concept of a single animal and a single tube can provide more accurate pre-clinical trial data. The pre-clinical results of this study will directly benefit LRBA-deficient patients who suffer severe CVID symptoms, even death and cannot be cured (Lopez-Herrera et al., 2012; Alangari et al., 2012; Burns et al., 2012). Thanks to the CRISPR technique, primate ko models can be generated (Niu et al., 2014). The all-in-one concept can be used to create primate models for pre-clinical trials and will dramatically reduce cost and animal number as only one animal is needed for each treatment group and can be used for its lifetime.

The animal model of the invention is highly innovative due to several features. The concept of phenotyping in a single animal and single tube will eliminate genetic, environmental and experimental variations that can contribute to or even mask the phenotype (Eisener-Dorman et al., 2009; Ridgway, 2014). In addition, flow cytometry can be used to analyze millions of cells with multiplex ability. Consequently, without the interference of variations, small differences in phenotype can be detected, allowing high resolution of the phenotyping required for those caused by less penetrance (more dependent on genetic background) of the targeted gene. As controls are not required, establishing a congenic strain is not necessary, the all-in-one model can be crossed with many strains and study Lrba in a wide genetic background required for mimicking genetic diversity in complex human CVID. F2 mice can be used directly for experiments. This can save tremendous time and money. The single animal concept is also very useful to study gene-gene interactions. Because if a significant difference is detected in B cell phenotypes from two animals either from the same or different inbred or outbred strain, it may indicate the presence of a modifier gene(s), which can be determined by the next generation sequencing of the whole exome from the two mice. Therefore, this model can be used to quickly discover gene-gene interactions, which are critical for studying complex human diseases. Knockout, overexpression and reporter mouse models are usually generated separately and only one model is studied in most labs due to time and financial limitations. With the all-in-one model of the invention, LRBA expression can be manipulated in multiple ways and the three genotypes can be generated in a single animal so the data therefore are more comparable.

The all-in-one animal model addresses a critical problem that current animal models cannot solve in modeling complex human immunodeficiency diseases that require great genetic variations present in the population. The Lrba gene has previously been cloned and a conditional knockout mouse model has been successfully produced (Wang et al., 2001; Wang et al., 2002). The all-in-one animal model provides the unique opportunity to facilitate high resolution study of the LRBA gene, a novel, unique and important immune regulator to understand a critical aspect of immunodeficiency. However, as indicated above, the all-in-one animal model may also be utilized for high resolution study of any gene.

There are alternative ways in which the all-in-one animal model can be produced, including alternative ways to switch the gene of interest on and off. An alternative to the CRISPR method, the traditional knock-in method based on mouse embryonic stem cells can be used to obtain this model. Similar to the Cre-loxP technology, Flp-FRT or PhiC31 Integrase-mediated recombination can also be used in the place of Cre-loxP recombination to turn on gene expression. In order to produce other species of animal models, the species-specific sequences will be used to replace mouse sequences. Other changes include, fertilized eggs and foster mothers from that species.

As an alternative to the fluorescent proteins (FP), Fluorogen activating peptide—FAP-tags®, a new class of small genetically encoded reporters that exhibit fluorescence only in the presence of micromolar concentrations of particular nontoxic soluble fluorogens, can be used in the place of FPs (http://spectragenetics.com/).

A vast array of experiments can be carried out using the all-in-one animal model, in a general step-by-step fashion, including experiments that would be conducted on the animal itself and on the cells (e.g., B cells) obtained from the animal.

1. Recapitulation of LRBA deficiency on the development of lymphocytes. In addition, LRBA is expressed ubiquitously, especially in hematopoietic cells and stem cells [13,15]. The inventors will use the multiparametric flow cytometry developed by BD Biosciences to study the development of B and T lymphocytes in this model. (a) Analysis of B-cell developmental stages in mouse bone marrow [77]. Seven different developmental phases can be discriminated in bone marrow by a panel of seven B cell surface markers. Pre-pro-B, Pro-B, and Pre-B cells can be distinguished within the low positive CD45R population based on their differential expression of BP1 and CD24. Immature, transitional, and early and late mature B cells could be segregated based on differential expression of IgM and IgD [77]. To study the effect of Lrba ko in the periphery, mature B cell presence in mouse spleens will also be determined. (b) Analysis of T-cell developmental stages in thymus [78]. The six developmental stages, four double-negative (DN1, DN2, DN3, DN4), double-positive (DP) and single-positive (SP), can be discriminated in thymus by an 8-color panel of cell surface markers. B220 negative events will be gated first, and then CD4 vs CD8 will identify the DN, DP, and SP cell populations. CD44 vs CD25 will identify the four substages (DN1 to DN4) in DN cell population. The low, intermediate, and high expression levels of TCR β corresponds to DN, DP and SP cells. CD69 and CD5 will be included in the panel because they are indicators for positive selection and the intensity of TCRs and self MHC-peptides interactions. Mature T cell presence in mouse spleens and lymph nodes will also be determined to study the effect of Lrba ko in the periphery. The activation state of B cells or T cells will also be determined in the periphery using MHC class II, CD40 and CD86 for B cells and, CD44, CD62L and CD25 for T cells, respectively. *Flow cytometry methodologies: LSR-II flow cytometer, which has an analysis rate of up to 40,000 cells per second, and the capacity to measure 15 cell markers. 40 samples can be analyzed within a couple of hours. 1) To exclude cell aggregates, two sequential gates of scatter width vs height signals will be applied. The singlet population will then be gated by forward scatter vs side scatter to exclude dead cells and debris. “Live cells” will be gated using a contour plot and then switch to a dot plot for easy monitoring of acquisition. 2) A FMO-control (Fluorescence Minus One) is a control sample composed of all antibody labels except one, and will be used as a negative control in place of an isotype control for that antibody staining. 3) All antibodies will be titrated. 4) The fluorophores used by BD that overlap with iRFP of CFP will be replaced with other fluorophores. (c) Others: 1) The One Step Staining Mouse Treg Flow™ Kit (BioLegend) will be used to detect Treg cells in mouse spleens. 2) Plasma antibody isotyping (IgG1, IgG2a, IgG2b, IgG3, IgA and IgM) will be performed using the Pierce Rapid ELISA Mouse mAb Isotyping Kit. 3) Lymph nodes, lung and intestine sections will be analyzed by fluorescent microscopy [72] to examine lymphoproliferation and lymphocyte infiltration. 4) Comprehensive standardized gross and histopathologic analyses will be performed, including the analyses of organ weights, serum chemistries and hematology. The inventors will use 12 age-matched mice of both sexes, 6 mutant and 6 wild-type controls for the analyses.

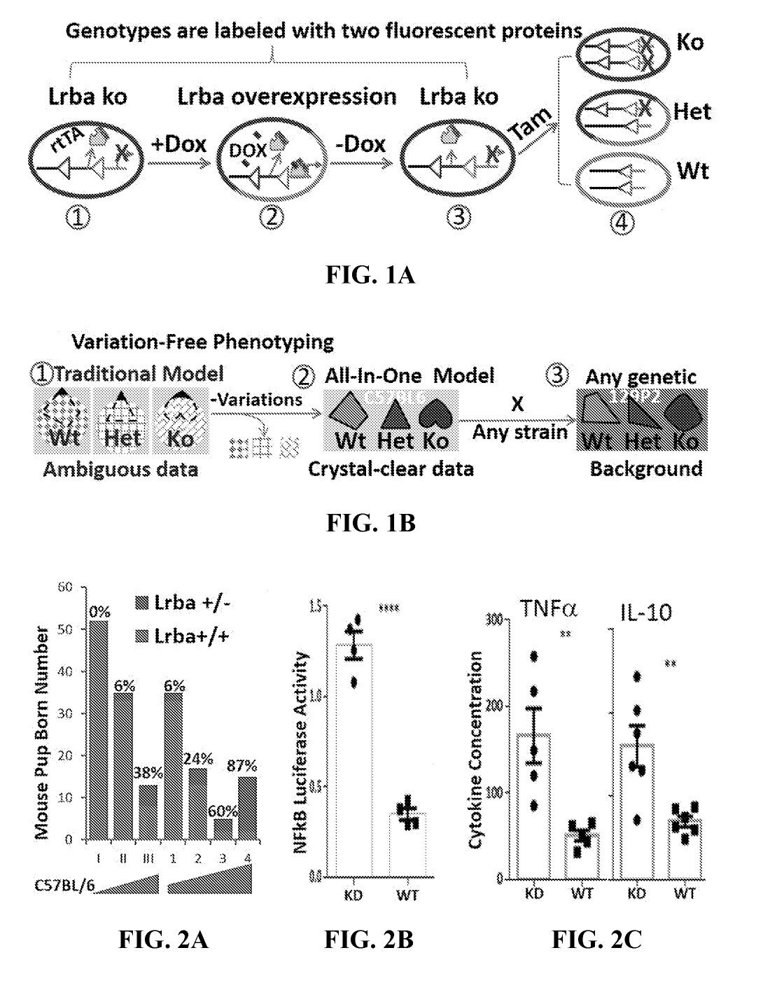

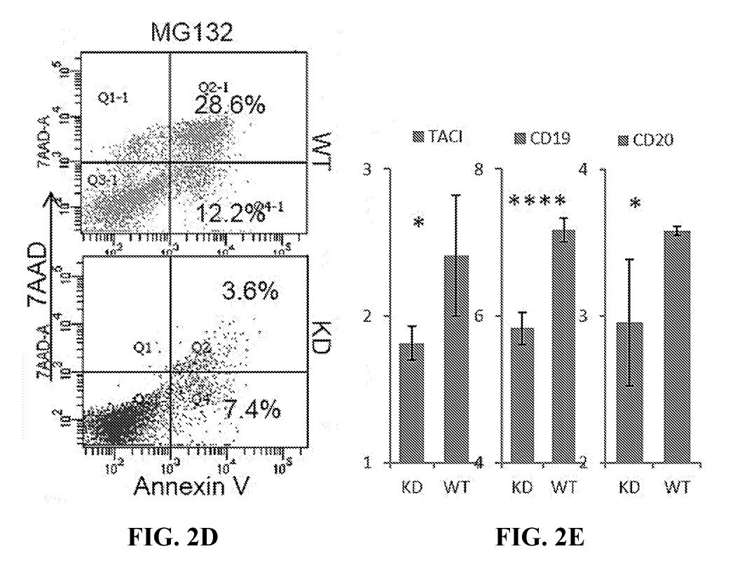

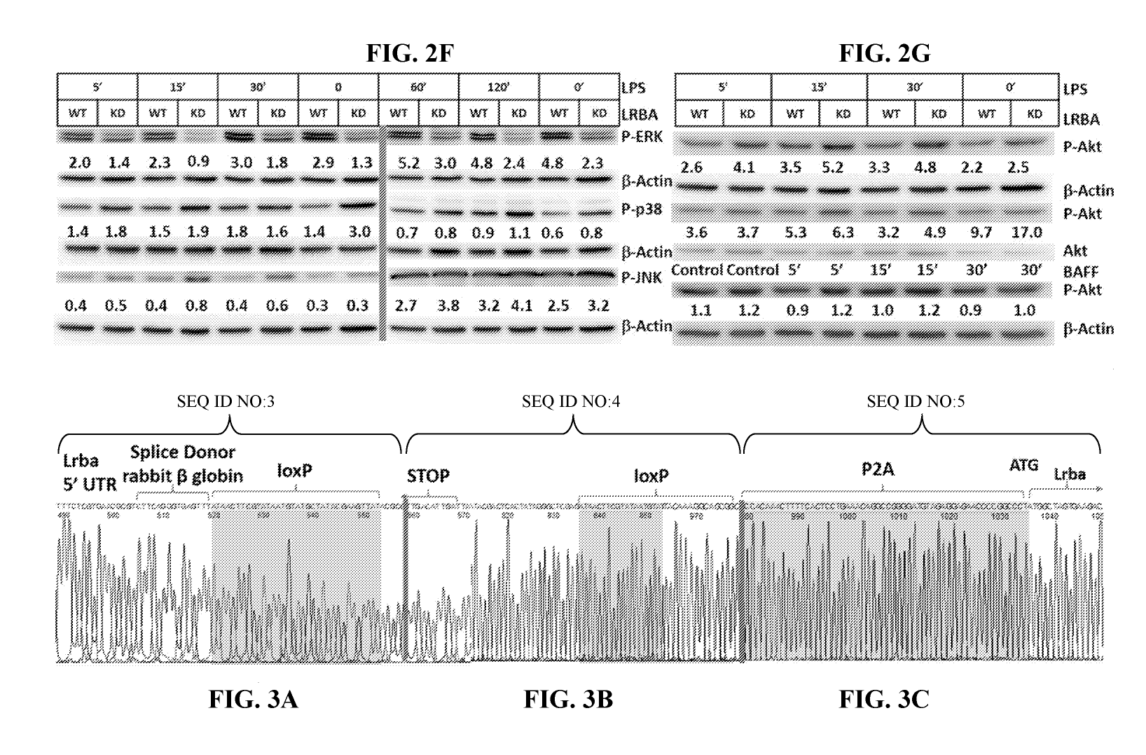

2. Recapitulation of LRBA deficiency on different genetic backgrounds: The phenotype is different in the two strains (FIG. 2A). The CreERT2 mice will be mated with 129P2 mice, and then the same set of experiments will be carried out. Results from the two genetic backgrounds will then be compared to find background-specific phenotypes (FIG. 1) to to identify modifiers.

The impact of LRBA deficiency on the development of B or T cells at each stage has not been investigated yet. The all-in-one animal model will allow highly sensitive detection of any abnormalities in B and T cell development caused by Lrba deficiency, which may not be detectable with the current techniques. 1) Based on human data, it is expected, as shown in FIG. 4C, that the percentage of immature lymphocytes will follow the order of Ko>Het>Wt, at each developmental stage, while the percentage of the mature cells including Treg will have the reversed order of Ko<Het<Wt. However, this prediction is based on the intrinsic effects of Lrba. It is possible that different types of cells can affect each other by trans-effects. To distinguish cell intrinsic-effects from trans-effects, similar experiments will be carried out but with variable starting percentages of the three types of cells by treating the CreERT2 mice with the TAM-treating conditions as determined above. The correlation coefficients of the results will be calculated to determine if there are any trans-effects [79]. 2) It is assumed that the Cre recombination efficiency is similar in the bone marrow and in the periphery. To increase accuracy, the Cre recombination efficiency in the bone marrow will be detected to establish a correlation between the TAM treatment condition and the recombination efficiency. 3) It is expected that some phenotypes are different; others are the same on the two backgrounds. The new phenotypes can be isolated and stabilized by further backcrossing, then WGS will be used to identify modifiers. 4) It is expected that the FP expression will respond to Dox. After Tam treatment, it is expected that the three genotypes, i.e. wt, het, and ko can be identified in B cells in the same CreERT2. 5) Plasma antibodies of Lrba deficient mice are expected to be lower than that of wt mice. 6) lymphoproliferation and lymphocyte infiltration may be observed in lymph nodes, lung and intestine sections.

3. Linage tracing of hematopoietic cells. The three genotypes can be induced at early development of hematopoietic cells, e.g. hematopoietic stem cells, then trace the development of each genotype using flow cytometry and fluorescent microscopy, and answer the questions whether different genotypes have different development consequences in terms of cell numbers, cell types (B, T lymphocytes, monocytes, dendritic cells) and cell subtypes (CD4, CD8 T cells, B1 and B2 B cells, etc.).

4. Study lymphocyte and stromal cell interaction. Lymphocytes undergo massive cell death at multiple developmental stages in order to eliminate non- or self-reactive lymphocytes through positive and negative selection, in which stromal cells play an important role. Since both lymphocytes and stromal cells are labeled with fluorescent colors specific to the genotypes, the influence of the different genotype of the stromal cells on the selection of lymphocytes can be studied.

5. Study the cell-cell interactions of hematopoietic cells. Similarly, the cell-cell interaction play an important role in the activation or inhibition of hematopoietic cells, such as the activation of B cells by T cells, the inhibition of T cell proliferation by regulatory T cells.

Example 1 describes how to generate animal model of the invention (an all-in-one mouse model) in which Lrba expression can be turned on/off in a spatiotemporal and trackable manner. A transcription control cassette (TCC) composed of a CAG promoter, STOP, tet-inducible system, two FP genes and three loxP sites will be inserted into the Lrba genomic locus by the CRISPR technique (Zhou et al., 2014; Wang et al., 2013; Yang et al., 2013; Fujii et al., 2013).

Example 2 describes determination of whether Lrba regulates the cell membrane levels of the CVID-associated receptors, in order to test if the all-in-one animal model is working and the novel idea to study simultaneously multiple phenotypes in a single mouse and single tube (FIG. 1).

Following are examples that illustrate procedures for practicing the invention. These examples should not be construed as limiting. All percentages are by weight and all solvent mixture proportions are by volume unless otherwise noted.

### EXAMPLE 1

### Generation of an All-in-One Mouse Model in which Lrba Expression can be Turned On/Off in a Spatiotemporal and Trackable Manner

CRISPR can induce up to 78% targeted mutation in mice with higher germline transmission and lower off-target mutation rate, greatly reducing time from years to months (Zhou et al., 2014; Wang et al., 2013; Yang et al., 2013; Fujii et al., 2013; Yang et al., 2013; Jinek et al., 2012). It causes DNA double strand break (DSB), which is usually repaired by the non-homologous end-joining (NHEJ) repair pathway, which results in deletion or insertion mutations. It also can be repaired by homology-directed-repair (HDR). A DNA fragment flanking by homologous sequences can be inserted into the cleaved site based on homologous recombination, the efficiency of which can be 5000 times higher than traditional homologous recombination (Donoho et al., 1998). Due to these advantages, the inventors plan to use the CRISPR/HDR technique to knock-in the transcription control cassette (TCC) into the Lrba genomic locus in order to generate the all-in-one mouse model and generate four mouse lines (FIG. 4B). Bearing similarity to this strategy, a loxP-STOP-TRE-loxP cassette (3.5 kb) has been successfully inserted and generated multiple lines (Tanaka et al., 2010). Other TCC advantages include 1) the Tet-On third generation tet-inducible gene expression system which is 100-fold more dox-sensitive than the original TetOn system and has significantly reduced background and is widely cited (Zhou et al., 2006; Loew et al., 2010; Kistner et al., 1996); 2) FP reporter genes: iRFP670 RFP is a near-infrared fluorescent protein that can be used for in vivo imaging for low background signals and cytometry (Shcherbakova and Verkhusha, 2013); Aquamarine CFP is superior to the popular form ECFP (Erard et al., 2013). Both genes were codon optimized and synthesized by IDTDNA and are used to track Lrba promoter activity and Lrba expression by flow cytometry. 3) P2A peptide is used to link two proteins, RFP-2A-rtTA, CFP-2A-Lrba to be co-expressed by a single promoter (Kim et al., 2011). Due to small size (57 bp) and highly self-cleavability and ability to produce equal molar ratio of the two proteins, 2A is superior to IRES, which is large and causes differential expression of the two genes it links. 4) Splice sites are added to avoid ineffective translation of a CDS due to sequences potentially containing ATG, long UTR and secondary structure added before that CDS.

1. Generation of Tri-lox (α) Mice by CRISPR.

Generation of Tri-lox (α) mice by CRISPR will be done as previously described (Yang et al., 2013). Briefly, T7 promoter will be added to the Cas9 coding sequence (CDS) and Lrba sgRNA by PCR using the primer pairs (Yang et al., 2013). The RNAs will be synthesized by T7 RNA polymerase and purified. Cas9 mRNA (100 ng/ml), sgRNA (50 ng/ml) and 200 ng/ml target plasmid DNA will be injected into the fertilized B57BL/6 eggs. The genomic DNA from targeted and control mice, age 8 to 12 days, will be extracted from clipped toes and used for PCR screening: The correct 5′ and 3′ end targeting will be confirmed by long range PCR protocol (Wang et al., 2002) using the primers from the vector and the genomic DNA sequence outside of the short or the long arms (FIG. 4A). The PCR products are predicted to be 3.6 kb and 9.3 kb. The mice that are correctly targeted as identified by PCR will be confirmed by Southern blot with the probes shown in FIG. 4A: For the 5′ targeting end, EcoRI-digested genomic DNA will be hybridized with a 5′ external probe with expected fragment sizes of 5 kb (wt) and 7.6 kb (targeted, T). The blot will then be stripped and hybridized with neo internal probe with expected fragment sizes of none (wt) and 7.6 kb (T). Similarly, for the 3′ end, SfiI-digested genomic DNA will produce 25 kb (wt) and 17 kb (ko) bands using a 3′ external probe. The stripped blot will be hybridized with an internal probe with expected fragment sizes of none (wt) and 15 kb (T). Internal vector probes will be used to detect illegitimate insertions.

2. Generation of Multiple Lines by Cre Recombination.

The correctly targeted mice will be mated with C57BL/6 mice to obtain F1 germline transmission mice (FIG. 4B) (Kranz et al., 2010). The potential mutations of the off-target sites identified by using the CRISPR Design tool will be further identified by PCR/sequencing. Lrba targeted mice without off-target mutations will be used as the founders to establish the colony. The general Cre deleter (JAX#006054) will be used to produce α, β, γ, and δ mouse strains (FIG. 4B) since Cre mediated recombination is normally incomplete, i.e., the tri-flox will produce mono-flox (δ), two di-flox (β and γ) (Wang et al., 2002; Leneuve et al., 2003). Briefly, the Cre male will be mated with the Lrba α/α female. The female offspring will have Cre (X-linked) (Su et al., 2002) and will be mated with C57BL/6 wt mice. Germ cells mosaic, i.e., some with one loxP, some two loxP and some three loxP sites, resulting from incomplete Cre recombination will produce desired mice identified by PCR genotyping (Primers P1, P2 and P3) and flow cytometry of peripheral blood (FP colors) (FIG. 4B). Although α and β mice will have white blood cells expressing RFP and γ and δ mice have cells expressing CFP, PCR genotyping will produce different product sizes to distinguish them since α and γ mice have two loxP sites between the two primers, and β and δ mice have only one loxP site (FIG. 2B). Homozygous strains will be produced by mating the Lrba heterozygous mice with the same knockin (kn) configuration.

It is expected that the all-in-one mouse model and its three derivatives will be successfully generated and can be used to study Lrba in a spatiotemporal and trackable manner in a single mouse and single tube with high resolution, so that the genetic diversity in the human population can be mimicked to facilitate gene-gene interactions to better understand and treat complex human diseases associated with deregulated LRBA. This model can be made available to scientists who are interested in LRBA and LRBA regulated genes, such as NFκB, MAPKs, AKT, TNFα, IL10 and CVID receptors, and EGFR, NOTCH and RAS/MAPK pathways critical in CVID, autoimmunity and basic immunology. One potential concern is that the CRISPR targeting may induce off-target mutations (Yang et al., 2013; Fu et al., 2013). To detect any such potential mutations, the top 20 genome-wide off-target sites identified by the CRISPR Design tool will be amplified by PCR and sequenced. If mutations are detected, then the founders will be backcrossed with C57BL/6 mice multiple times until no mutations can be detected. Another potential concern is that since the kn fragment is large, 5.9 kb, the CRISPR/HDR one-step targeting rate may be low. The targeting rate is usually one in 20 for the CRISPR/HDR, which has been successfully used to knock-in a 3 kb transgene cassette (Yang et al., 2013). If this targeting fails, as thousands of ES clones can be screened by PCR, and double strand break (DSB) can increase targeting efficiency by 5000 fold (Donoho et al., 1998), the inventors will use the traditional ES cell gene targeting procedure combined with the CRISPR technique to obtain the targeted ES cells to obtain kn mice. An alternative to ES cells is to test three different sgRNA to obtain the highest DSB rate in ES cells.

### EXAMPLE 2

### Determination of Whether Lrba Regulates the Cell Membrane Levels of the CVID-Associated Receptors

Vesicle trafficking is required for homeostasis of membrane receptors through cell membrane deposition, oligomerization, phosphorylation, internalization, recycling and degradation (Wiley, 2003). As a vesicle trafficking regulator, LRBA may regulate CVID cell membrane receptors. LRBA regulates two cell membrane receptors: epidermal growth factor receptor (EGFR) and NOTCH (Wang et al., 2004; Yatim et al., 2012; Volders et al., 2012). Three CVID receptors CD19, CD20 and BAFFR are downregulated in LRBA deficient patients (Lopez-Herrera et al., 2012). The preliminary data show that LRBA knockdown down-regulates CVID receptors in vitro. The inventors' working hypothesis is that LRBA regulates the cell surface levels of CVID receptors on B cells in vivo. The inventors will use the β kn mice labeled as Lrba-β/β which express rtTA (FIG. 4B) by Lrba promoter but do not express Lrba due to the STOP cassette placed before the translation start codon ATG. Lrba expression can be simply turned on in the presence of Dox and turned off by withdrawing Dox. The phenotypes can be studied before adding Dox (Lrba negative), after adding Dox (Lrba positive) and after Dox withdrawal (Lrba negative) in a same mouse. Further within the same mouse, mosaic B cell populations with the three genotypes (wt, het and ko) can be generated by incomplete recombination mediated by Cre (FIGS. 4A and 4B). The phenotypes can be compared among the three populations in a single mouse.

1. Generation of Lrba-β/β creERT+/− mice: As shown in FIG. 1, the Cre gene that can be activated in the presence of tamoxifen (Tam) will be introduced into the β mice by mating with the CAG-creERT mice (JAX#019102) that ubiquitously express a Tam-inducible Cre recombinase to obtain Lrba-β/β creERT+/− mice.

2. Dox-induced Lrba gene expression. The Lrba-β/β creERT+/− mice and wt C57BL/6 mice (male, 6 wks of age) will be treated with or without Dox. rtTA will activate Lrba expression in Lrba-β/β creERT+/− mice in the presence of Dox. This kn switch will allow for endogenous expression of Lrba which can be turned on (+Dox) or off (−Dox).

3. Tam-induced Cre mosaic recombination. Three types of B cells (FIG. 4B. Lrba-β/β ko, red; Lrba-β/δ het, red and cyan; Lrba-δ/δ wt, cyan) will be produced in the same Lrba-β/β creERT+/− mouse by Tam-induced Cre mosaic recombination (FIGS. 1, 4A, and 4B) and can be distinguished by flow cytometry: Lrba-β/β B cells are red due to RFP expression; Lrba-β/δ B cells are red and cyan due to RFP and CFP expression; Lrba-δ/δ B cells are cyan due to CFP expression. The conditions (dosing and time) of Tam treatment will be optimized to obtain roughly equal numbers of the three B cell types, as determined by flow cytometry: Lrba-β/β mice will receive 0, 3, or 9 mg of Tam (ip injection) for 1 to 5 consecutive days. Then, on day 24, the mice will be treated with Tam (i.p.) at the optimized condition to induce partial Cre recombination producing three types of B (Hayashi and McMahon, 2002). On days 0, 10, 17 and 31, blood from the submandibular vein will be collected and subjected to flow cytometry (FIG. 5).

4. Flow cytometry: As mouse peripheral blood is limited, the inventors will use a no-lyse, no-wash staining flow cytometry technique using 20 μL of whole blood for each analysis (Weaver et al., 2002; Weaver et al., 2010). The B cells in the peripheral blood will be subjected to multiparametric flow cytometry of the CVID receptors using mouse-specific antibodies against BAFFR, TACI, CD19, CD20, CD21 and CD81. Two panels will be used and live/dead discrimination will be determined using DAPI or 7AAD. Data acquisition will be performed. At least 10,000 cells will be collected using an LSRII flow cytometer then analyzed with FACSDiva software with the gating strategy: the first gating will exclude cell debris based on a forward scatter/side scatter plot, the second gating will exclude non-B220 (pan B-cell marker) cells, and the third gating will be for RFP and CFP.

It is expected that the mouse model will work as shown in FIG. 1, without Dox, there is no Lrba expression (see \textcircled{1} in FIG. 1) detected by realtime PCR and Western blot and the cells are red (RFP); with Dox, Lrba and CFP will be expressed, the cells are cyan (see \textcircled{2} in FIG. 1) and so on. The cell membrane levels of the CVID receptors will be increased after Lrba is induced in Lrba-β/β mice by Dox at day 10 compared to the mice before treatment (−Dox). The levels will be dropped back to “baseline levels” after Dox withdrawal, i.e., no difference among these Lrba-β/β ±Dox mice, but will be lower than wt. After Tam treatment, it is expected that three genotypes, i.e., β/β, β/δ, and δ/δ will be identified in B cells in the same β/β mice. The levels of the CVID receptors will be higher in δ/δ but lower in β/β with β/δ in between. This will demonstrate that LRBA regulates CVID receptors in vivo. LRBA deficiency may exert its effects on immunodeficiency and autoimmunity through deregulating these receptors. 1) The no-lyse, no-wash staining flow cytometry technique uses 10 time less volume than a a regular cytometry protocol. If it does not work well, the inventors will use a regular cytometry protocol, which will require greater blood volume. Therefore, the inventors will use older mice (>6 months). 2) The Cre expression of creERT^{+/-} mice may be somewhat leaky, resulting in constitutive recombination before Tam induction (Elefteriou and Yang, 2011). If the inventors cannot generate the three genotypes in a single mouse due to this problem, the Mx-Cre mice will be used (JAX#002527) (Kuhn et al., 1995) and will induce Cre expression by ip injection of polyinosinic-polycytidylic acid.

Prelimary Results: LRBA Knockdown (KD) Upregulates NFκB, AKT, P38, JNK, TNFα and IL-10 but Downregulates ERK and CVID Receptors.

LRBA is LPS-responsive and an anchoring protein for protein kinase A (PKA) (Wang et al., 2001; Kerr et al., 1996). LPS and PKA can activate NFκB, which is implicated in the pathogenesis of human immunodeficiency diseases (Kurylowicz and Nauman, 2008). This NFκB signaling may be affected by LRBA deficiency. The results show LRBA KD increases NFκB activity in a dose-dependent manner (FIG. 2A), and increases TNFα and IL10 (FIG. 2B) but downregulates CVID receptors (FIG. 2C). Like NFκB, MAPKs and AKT are critical downstream hubs of the TLR4/LPS signal transduction pathway (Oeckinghaus et al., 2011). LRBA KD downregulates ERK but upregulates p38, JNK (FIG. 2D) and AKT (FIG. 2E). Complementing the preliminary data, Wech and Nagel reported decreased MAPK (ERK orthologue) and increased JNK upon rugose (LRBA orthologue) mutation (Wech and Nagel, 2005).

Target Vector and Single Guide RNA (sgRNA)/Cas9 Vector Construction.

The target vector was constructed from BAC a clone using homologous recombineering (HR). The subsequent engineering of the vector included multiple rounds of HR (Quick & Easy Conditional Knock Out Kit), Seamless Ligation Cloning Extract (SLICE) and traditional ligation. The correct cloning of the target vector was characterized by restriction digestion (FIGS. 3A-3C) and confirmed by sequencing the TCC and the junctions between the 3′ arm/5′ arm/TCC/backbone regions. Portions of sequence traces around loxP sites show accurate cloning (FIGS. 3G-3I). A secretable luciferase gene was inserted in place of Lrba (FIG. 3B) and used to show the tet-inducible system in the TCC function as expected (FIG. 3D). Further, the inventors will test the δ (FIG. 4B) equivalent luciferase vector (FIG. 3C) and will obtain β and γ (FIG. 4B) equivalent luciferase vectors and test them work properly before going to CRISPR egg injection. The neo cassette in the target vector is not required by CRISPR and will be removed by pE-FLP resulting in 5.9 kb TCC. The sgRNA sequence was designed using the online CRISPR Design Tool and cloned into the pX330 vector (Cong et al., 2013). As the sgRNA sequence spans the insertion site, the target vector does not have the sequence and will not be cleaved by the Cas9 nuclease. The sgRNA sequence also has a SpeI site and was used to identify gene mutations (FIG. 3E), in addition to a T7 Endonuclease I (T7EI) assay (FIG. 3F). Both assays indicate that the Lrba locus has been successfully mutated in the ES cells.

LRBA gene deficiency causes severe primary immunodeficiency disease, which is manifested as multiple diseases with highly variable symptoms, such as common variable immunodeficiency (CVID), FOXP3 deficiency-like syndrome, autoimmune lymphoproliferative syndrome, inflammatory bowel diseases etc. LRBA overexpression is found in multiple cancers, and it is a molecular signature for breast cancer mortality and recurrence. In addition, LRBA mutations are present in more than 15% of CVID patients. This indicates that there are many unknown genes interacting with the LRBA gene to cause so many different symptoms and diseases.

The severity and prevalence of LRBA mutations make LRBA one of the most important genes related to human health. The two basic functions of LRBA, regulation of cell survival and regulation of vesicle trafficking, are central to the development of lymphocytes, the deregulation of which is the major cause of autoimmunity and immunodeficiency. Complementally, LRBA regulates multiple critical immune regulators. Moreover, LRBA deficient patients have highly variable symptoms, indicating that there are many unidentified genes (modifiers) which interact with LRBA. However, none of these genes' roles in the pathology of LRBA deficiency is yet known. The long-term goal is to identify these critical immune regulators and to study how LRBA interacts with them in vivo to cause specific phenotype in order to better understand this disease. However, there are two critical barriers to progression in this field. 1) The genetic, epigenetic and environmental variations between mice often mask the phenotype of a gene, resulting in ambiguous results. That no mouse model of LRBA deficiency has been described likely reflects the difficulty of modeling this disease with the current techniques. 2) LRBA human “knockouts” show that the phenotype of LRBA is highly dependent on genetic background. To replicate all phenotypes seen in human “knockouts” in mice, an existing target mouse line should be backcrossed with many mouse strains to establish many congenic strains. This is impossible due to cost and time as it takes ˜3 years to change a genetic background, and there are still variations that may mask phenotypes.

The all-in-one animal model of the invention will overcome two critical barriers to modeling this complex human disease (as well as others) by using a variation-free phenotyping technique to quickly identify these LRBA-interacting genes, so that a clear correlation between the phenotype and genotypic can be determined in order to better understand the disease.

Generation of an all-in-one animal model in which wild type, heterozygote, and knockout lymphocytes can be produced in a single animal, and the three genotypes are labeled with three different fluorescent colors will overcome these problems. Phenotypes can thus be studied by flow cytometry in a single tube with high sensitivity due to the elimination of variations. Furthermore, since each genotype will have the other two genotypes as controls in a single animal, the F1 mice can be used directly to identify genetic modifiers. This can significantly save in time and cost. Thus, it is possible now that Lrba (or other genes) can be studied on various genetic backgrounds, which is required for recapitulating this complex human disease. Recapitulating the key features of LRBA deficient disease is important. The impaired lymphocyte development is one of the most important features. The all-in-one animal model will allow highly sensitive detection of any abnormalities in lymphocyte development.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Alangari, A. et al. LPS-responsive beige-like anchor (LRBA) gene mutation in a family with inflammatory bowel disease and combined immunodeficiency. J Allergy Clin Immunol 130, 481-8 e2 (2012).
Andres, S. A., Brock, G. N. & Wittliff, J. L. Interrogating differences in expression of targeted gene sets to predict breast cancer outcome. BMC Cancer 13, 326 (2013).
Burns, S. O. et al. LRBA gene deletion in a patient presenting with autoimmunity without hypogammaglobulinemia. J Allergy Clin Immunol (2012).
Cong, L. et al. Multiplex genome engineering using CRISPR/Cas systems. Science 339, 819-23 (2013).
Cullinane, A. R., Schaffer, A. A. & Huizing, M. The BEACH Is Hot: A LYST of Emerging Roles for BEACH-Domain Containing Proteins in Human Disease. Traffic (2013).
Donoho, G., Jasin, M. & Berg, P. Analysis of gene targeting and intrachromosomal homologous recombination stimulated by genomic double-strand breaks in mouse embryonic stem cells. Mol Cell Biol 18, 4070-8 (1998).
Eibel, H., Salzer, U. & Warnatz, K. Common variable immunodeficiency at the end of a prospering decade: towards novel gene defects and beyond. Curr Opin Allergy Clin Immunol 10, 526-33 (2010).
Eisener-Dorman, A. F., Lawrence, D. A. & Bolivar, V. J. Cautionary insights on knockout mouse studies: the gene or not the gene? Brain Behav Immun 23, 318-24 (2009).
Elefteriou, F. & Yang, X. Genetic mouse models for bone studies—strengths and limitations. Bone 49, 1242-54 (2011).
Erard, M. et al. Minimum set of mutations needed to optimize cyan fluorescent proteins for live cell imaging. Mol Biosyst 9, 258-67 (2013).
Fu, Y. et al. High-frequency off-target mutagenesis induced by CRISPR-Cas nucleases in human cells. Nat Biotechnol 31, 822-6 (2013).
Fujii, W., Kawasaki, K., Sugiura, K. & Naito, K. Efficient generation of large-scale genome-modified mice using gRNA and CAS9 endonuclease. Nucleic Acids Res 41, e187 (2013).
Gathmann, B., Binder, N., Ehl, S., Kindle, G. & Party, E. R. W. The European internet-based patient and research database for primary immunodeficiencies: update 2011. Clin Exp Immunol 167, 479-91 (2012).
Hayashi, S. & McMahon, A. P. Efficient recombination in diverse tissues by a tamoxifen-inducible form of Cre: a tool for temporally regulated gene activation/inactivation in the mouse. Dev Biol 244, 305-18 (2002).
Jinek, M. et al. A programmable dual-RNA-guided DNA endonuclease in adaptive bacterial immunity. Science 337, 816-21 (2012).
Kerr, W. G., Heller, M. & Herzenberg, L. A. Analysis of lipopolysaccharide-response genes in B-lineage cells demonstrates that they can have differentiation stage-restricted expression and contain SH2 domains. Proc Natl Acad Sci USA 93, 3947-52 (1996).
Kholodenko, B. N. Four-dimensional organization of protein kinase signaling cascades: the roles of diffusion, endocytosis and molecular motors. J Exp Biol 206, 2073-82 (2003).
Kim, J. H. et al. High cleavage efficiency of a 2A peptide derived from porcine teschovirus-1 in human cell lines, zebrafish and mice. PLoS One 6, e18556 (2011).
Kistner, A. et al. Doxycycline-mediated quantitative and tissue-specific control of gene expression in transgenic mice. Proc Natl Acad Sci USA 93, 10933-8 (1996).
Kranz, A. et al. An improved Flp deleter mouse in C57Bl/6 based on Flpo recombinase. Genesis 48, 512-20 (2010).
Kuhn, R., Schwenk, F., Aguet, M. & Rajewsky, K. Inducible gene targeting in mice. Science 269, 1427-9 (1995).
Kurylowicz, A. & Nauman, J. The role of nuclear factor-kappaB in the development of autoimmune diseases: a link between genes and environment. Acta Biochim Pol 55, 629-47 (2008).
Leneuve, P. et al. Cre-mediated germline mosaicism: a new transgenic mouse for the selective removal of residual markers from tri-lox conditional alleles. Nucleic Acids Res 31, e21 (2003).
Loew, R., Heinz, N., Hampf, M., Bujard, H. & Gossen, M. Improved Tet-responsive promoters with minimized background expression. BMC Biotechnol 10, 81 (2010).
Lopez-Herrera, G. et al. Deleterious mutations in LRBA are associated with a syndrome of immune deficiency and autoimmunity. Am J Hum Genet 90, 986-1001 (2012).
Niu, Y. et al. Generation of gene-modified cynomolgus monkey via Cas9/RNA-mediated gene targeting in one-cell embryos. Cell 156, 836-43 (2014).
Oeckinghaus, A., Hayden, M. S. & Ghosh, S. Crosstalk in NF-kappaB signaling pathways. Nat Immunol 12, 695-708 (2011).
Park, M. A., Li, J. T., Hagan, J. B., Maddox, D. E. & Abraham, R. S. Common variable immunodeficiency: a new look at an old disease. Lancet 372, 489-502 (2008).
Podjasek, J. C. & Abraham, R. S. Autoimmune cytopenias in common variable immunodeficiency. Front Immunol 3, 189 (2012).
Ridgway, W. M. A new tool for dissecting genetic control of type 1 diabetes. Diabetes 63, 56-8 (2014).
Shamloula, H. K. et al. rugose (rg), a Drosophila A kinase anchor protein, is required for retinal pattern formation and interacts genetically with multiple signaling pathways. Genetics 161, 693-710 (2002).
Shcherbakova, D. M. & Verkhusha, V. V. Near-infrared fluorescent proteins for multicolor in vivo imaging. Nat Methods 10, 751-4 (2013).
Su, H., Mills, A. A., Wang, X. & Bradley, A. A targeted X-linked CMV-Cre line. Genesis 32, 187-8 (2002).
Tanaka, K. F. et al. Flexible Accelerated STOP Tetracycline Operator-knockin (FAST): a versatile and efficient new gene modulating system. Biol Psychiatry 67, 770-3 (2010).
Vogel, G. Genetics. Scientists dream of 1001 complex mice. Science 301, 456-7 (2003).
Volders, K. et al. Drosophila rugose is a functional homolog of mammalian Neurobeachin and affects synaptic architecture, brain morphology, and associative learning. J Neurosci 32, 15193-204 (2012).
Wan, Y. Y. & Flavell, R. A. Identifying Foxp3-expressing suppressor T cells with a bicistronic reporter. Proc Natl Acad Sci USA 102, 5126-31 (2005).
Wang, H. et al. One-step generation of mice carrying mutations in multiple genes by CRISPR/Cas-mediated genome engineering. Cell 153, 910-8 (2013).
Wang, J.-W. & Lockey, R. F. Lipopolysaccharide-responsive beige-like anchor (LRBA), a novel regulator of human immune disorders. Austin Journal of Clinical Immunology 1, 9 (2014).
Wang, J. W. et al. Deregulated expression of LRBA facilitates cancer cell growth. Oncogene 23, 4089-97 (2004).
Wang, J. W. et al. Influence of SHIP on the NK repertoire and allogeneic bone marrow transplantation. Science 295, 2094-7 (2002).
Wang, J. W., Howson, J., Haller, E. & Kerr, W. G. Identification of a novel lipopolysaccharide-inducible gene with key features of both A kinase anchor proteins and chs1/beige proteins. J Immunol 166, 4586-95 (2001).
Wang, T., Liu, N. S., Seet, L. F. & Hong, W. The emerging role of VHS domain-containing Tom1, Tom1L1 and Tom1L2 in membrane trafficking. Traffic 11, 1119-28 (2010).
Weaver, J. L., Broud, D. D., McKinnon, K. & Germolec, D. R. Serial phenotypic analysis of mouse peripheral blood leukocytes. Toxicol Mech Methods 12, 95-118 (2002).
Weaver, J. L., McKinnon, K. & Germolec, D. R. Phenotypic analysis using very small volumes of blood. Curr Protoc Cytom Chapter 6, Unit 6 30 (2010).
Wech, I. & Nagel, A. C. Mutations in rugose promote cell type-specific apoptosis in the Drosophila eye. Cell Death Differ 12, 145-52 (2005).
Wiley, H. S. Trafficking of the ErbB receptors and its influence on signaling. Exp Cell Res 284, 78-88. (2003).
Yang, H. et al. One-step generation of mice carrying reporter and conditional alleles by CRISPR/Cas-mediated genome engineering. Cell 154, 1370-9 (2013).
Yatim, A. et al. NOTCH1 nuclear interactome reveals key regulators of its transcriptional activity and oncogenic function. Mol Cell 48, 445-58 (2012).
Zhou, J. et al. Dual sgRNAs facilitate CRISPR/Cas9-mediated mouse genome targeting. FEBS J 281, 1717-25 (2014).
Zhou, X., Vink, M., Klaver, B., Berkhout, B. & Das, A. T. Optimization of the Tet-On system for regulated gene expression through viral evolution. Gene Ther 13, 1382-90 (2006).

### Supplemental Disclosure 1

The history of mouse genetics demonstrates that “clear and consistent phenotypes are the exception rather than the rule” [1]. Similarly, in human genetics, genetic risks have not been determined for the majority of common human diseases [2]. Both reflect the difficulty of defining a clear correlation between phenotype and genotype. 1) This is due to the fact that genetic and other experimental variations mask the phenotype of a gene [3,4]. The inventors thus propose a variation-free phenotyping method to increase phenotyping sensitivity so that “clear and consistent phenotypes are the rule” (FIG. 1B). 2) The human LRBA “knockouts” demonstrate that the phenotypes of LRBA knockout are highly dependent on genetic background. This indicates that there are unknown genes [5] interacting with LRBA to cause different symptoms or diseases, thus making phenotypes (Pn)=LRBA “knockout”+genetic backgrounds (Gn, n=1,2,3 . . . ). In order to replicate the P1 phenotype (e.g., Treg deficiency) of human “knockouts” in mice, the G1 background is required. Since the inventors do not know about the background G1 yet, the phenotype P1cannot be reproduced in mice. Even if a good phenotype (P1) of the ko mice is obtained, this existing target line should be backcrossed with many strains to recapitulate other Pn-1. Currently, this is impossible, as it takes ˜3 years to change a genetic background, and there are still variations that may mask phenotypes.

In contrast, animal and methods of the invention allow F1 mice to be used for phenotyping (FIG. 1B). Once a different phenotype is detected, the whole genome sequencing (WGS) can be used to identify the modifiers (Gn) [6]. If a different phenotype (P2) is observed after changing background from G1 to G2, the unknown modifier G2 will be able to be identified by WGS (FIG. 1B) [6]. To address any concern over “over-ambition”: 1) the inventors removed the “generation of multiple lines”; 2) the insert is now reduced to ˜4 kb so that mating with a Cre deleter is no longer required, saving several months of time; and 3) high-throughput flow cytometry will also be used to save time.

Genetic mosaics with different genotypes residing in the same organisms have been widely used to study biological processes [7]. Bearing some similarity, a loxP-STOP-TRE-loxP cassette has been used to control gene expression [8]. The inventors will experimentally test the utility of this model. Human LRBA and murine Lrba proteins have ˜94% homology. The phenotypes of a LRBA orthologue mutant in a fly can be rescued by a mouse LRBA-isoform transgene, demonstrating that LRBA function is highly conserved [9]. A goal is to overcome two critical barriers to generating a model that is truly representative of human diseases.

The deficiency of the LRBA gene causes primary immunodeficiency (PID), which is manifested as common variable immunodeficiency (CVID), FOXP3 deficiency-like syndrome [11] autoimmune lymphoproliferative syndrome (ALPS) [12], inflammatory bowel disease (IBD) etc. with highly variable symptoms in different patients. This indicates that genetic background plays an important role in determining phenotype (symptoms). The data also show that Lrba deficiency causes opposite phenotypes in different mouse strains. On the other hand, LRBA overexpression is found in multiple cancers [13] and is a molecular signature for breast cancer mortality and recurrence [14]. Moreover, LRBA mutations are present in more than 15% of CVID patients [15]. The severity and prevalence of LRBA mutations make LRBA one of the most important genes in human health. The inventors discovered two basic functions of LRBA, cell survival regulation [13] and vesicle trafficking regulation [5,68], which is required for the regulation of one-third of human proteins [16]. These functions are central to the development of lymphocytes, the deregulation of which is the major cause of autoimmunity and immunodeficiency. Supplemental to its functions, LRBA regulates multiple critical immune regulators [13] (FIGS. 2A-2G). Some of the data are supported by others' data in invertebrates [13,17,18]. The highly variable symptoms of LRBA deficiency also indicate that there are unknown genes [5] interacting with LRBA, i.e., phenotypes (Pn)=LRBA “knockout”+genetic backgrounds (Gn), which still is a “dark matter”: its role in the pathology of LRBA deficiency is currently unknown. The inventors' long-term goal is to identify these modifiers and delineate the molecular mechanism underlying LRBA deficient diseases.

However, the inventors were faced with two critical barriers to modeling this complex human disease. 1. The history of mouse genetics shows that “clear and consistent phenotypes are the exception rather than the rule” [1]. In the inventors' opinion, this is due to the fact that genetic, epigenetic, and environmental variations between intra-strain mice mask the phenotype of a gene, resulting in low sensitivity of phenotyping and thus ambiguous data. 2. It takes ˜3 years to change a genetic background, limiting the function study of a gene to one strain and making it impossible to use multiple strains. Each mouse strain is analogous to only a single human and thus lacks the genetic diversity required to identify genetic modifiers specific to different symptoms or diseases. To recapitulate human genetic variations in the population to find these modifiers, Lrba should be studied on a wide genetic background by backcrossing an existing target line with many mouse strains. That, to date, no mouse model of LRBA deficiency has been described by others likely reflects the difficulty of modeling this disease with the current techniques. To overcome these problems, the inventors propose to generate an all-in-one mouse model so that wild type (wt), heterozygote (het), and knockout (ko) cells can be produced and studied in a single mouse and tube without the interference of variations. This will allow for the identification of genetic modifiers through sensitive phenotyping and rapid genetic background change (FIGS. 1A-1B).

The inventors propose generating an all-in-one mouse model in which Lrba expression can be turned on/off in a spatiotemporal and trackable manner. A transcription control cassette (TCC) will be inserted into the Lrba genomic locus by the Clustered Regularly Interspaced Short Palindromic Repeats (CRISPR) technique. To test the functionality of the all-in-one mouse model in recapitulating the complex LRBA deficiency disease. The impact of Lrba deficiency on the development of lymphocytes will be determined in this model to provide a proof of principle for modeling complex immunodeficiency diseases in a single mouse.

The invention has the potential to solve the two critical barriers to modeling complex diseases, a task which requires sensitive phenotyping and great genetic diversity. The human LRBA and mouse Lrba genes have been cloned, a conditional knockout mouse model has been generated [10,16], and a novel CRISPR method for constructing a target vector has been developed [19]. These accomplishments ensure the successful generation of the all-in-one animal model for us as well as others with this unique opportunity to facilitate a highly sensitive study of the LRBA gene, a novel, unique immune regulator important in understanding a critical aspect of immunodeficiency, as well as other genes.

LRBA gene: 1) LRBA deficiency causes multiple diseases with highly variable symptoms in different patients [11,12,20-23]. The immune dysregulation, polyendocrinopathy, enteropathy, X-linked (IPEX)-like syndrome [11], autoimmune lymphoproliferative syndrome (ALPS) [12], common variable immunodeficiency (CVID) [20,21], inflammatory bowel diseases (IBD) [23], etc. were described in LRBA deficient patients. Some patients have early onset primary immunodeficiency (PID), while others initially present with autoimmunity but normal antibody levels, or IBD without other autoimmunity [23]. The same mutation causes hypogammaglobulinemia in one family but not in the other [12,21]. The highly variable symptoms in different patients indicate that genetic background plays an important role in determining phenotype (symptoms). The data also show that Lrba deficiency causes opposite phenotypes in different mouse strains (FIG. 2A). On the other hand, LRBA overexpression is found in multiple cancers [13], and is a molecular signature for breast cancer mortality and recurrence [14]. 2) LRBA likely has a high mutation frequency in the population due to its large genomic sequence (800 kb). LRBA mutations are present in more than 15% of CVID patients [15,23,24]. 3) The severity and prevalence of LRBA mutations make LRBA one of the most important genes in the understanding of the immune system and chronic immunological diseases. Two basic functions of LRBA were discovered, cell survival regulation [13] and vesicle trafficking regulation [5,68], which is required for the regulation of one-third of human proteins [16]. These functions are central to the development of lymphocytes, deregulation of which is the major cause of autoimmunity and immunodeficiency. Supplemental to its functions, LRBA regulates multiple critical immune regulators [13] (FIGS. 2A-2G). Some of the data are supported by others' data in invertebrates [13,17,18]. The highly variable symptoms of LRBA deficiency also indicate that there are unknown genes [5] interacting with LRBA, i.e. phenotypes=LRBA “knockout”+genetic backgrounds (genetic modifiers) or Pn=LRBAko+Gn (n=1,2,3 . . . ). Gn still is a “dark matter”: its role in the pathology of LRBA deficiency is unknown. The long-term goal is to identify these modifiers and delineate the molecular mechanism underlying LRBA deficient diseases.

The conventional mouse model technique has two major barriers to modeling complex human diseases. 1) The history of mouse genetics demonstrates that “clear and consistent phenotypes are the exception rather than the rule”, i.e. phenotypes cannot be detected in many knockouts [1]. The inventors propose this is due to the fact that genetic [25,26], epigenetic, and environmental variations between the intra-strain mice mask the phenotype of a gene, resulting in low sensitivity of phenotyping and thus ambiguous data [3,25-28]. These variations may result from the genes tightly linked to the targeted gene [26,27], single housing [29], or infections [30-37]. The phenotypes originally attributed to the targeted gene are actually caused by other genes [26,27]. 2) LRBA human “knockouts” show that the phenotype of LRBA is highly dependent on genetic background, which indicates that there are many unidentified genes (modifiers) interacting with LRBA to cause different diseases. To detect all phenotypes seen in human “knockouts”, an existing target mouse line should be backcrossed with as many strains as possible to establish multiple congenic strains so that these unknown modifiers contributing to different symptoms can be identified in order to understand LRBA deficient diseases [38-40]. This is currently impossible due to cost and time as it takes ˜3 years to change a genetic background. Moreover, mice from a congenic strain still have genetic variations in addition to other variations. No mouse model of LRBA deficiency has yet been described. This is unsurprising given that the symptoms of LRBA deficiency are highly variable, suggesting that some phenotypes may not present in one strain but may present in others and vice versa, and that the phenotype is masked by variations. This reflects the difficulty of modeling LRBA deficiency with the current techniques.

To overcome these problems, the inventors propose a variation-free phenotyping technique by generating an all-in-one mouse model, in which Lrba expression can be turned on/off in a spatiotemporal and trackable manner so that wt, het, and ko B cells with different FP colors can be produced in a single mouse. This will allow: 1) Highly sensitive phenotyping. Phenotypes can be studied by flow cytometry in a single mouse and in a single tube with high sensitivity as there will be no interference from genetic, epigenetic, environmental and procedure variations (FIGS. 1A-1B). This will allow the phenotypes to be attributed specifically to Lrba if they change in response to Lrba expression being switched on/off. 2) Rapid genetic background change to identify the modifier. With the animal model of the invention, each genotype will have the other two genotypes as controls in a single mouse. This will make using control mice and thus establishing congenic strains unnecessary. F1 mice can be used directly to identify genetic modifiers. Therefore, the proposed model can serve as a quick detector of modifiers (FIG. 1B). 3) A powerful tool to study the etiologies of common human diseases. The genome-wide association studies (GWAS) demonstrate that common diseases result from the complex interplay of many genes and environmental factors. In addition, they show that the impact of individual genes is small and can be masked by genetic and environmental variations. However, GWAS have failed to establish common variant risk for the majority of common diseases. The proposed method can be used to rapidly identify genes contributing to a common disease.

The proposed work is highly innovative due to the following key features: 1) The concept of phenotyping in a single mouse and single tube will eliminate genetic, environmental, and procedure variations that can contribute to or even mask the phenotype [26,27]. In addition, flow cytometry can be used to analyze millions of cells with multiplex ability. Consequently, without the interference of variations, small differences in phenotype can be detected, allowing for high resolution of the phenotyping required for those phenotypes that are caused by less penetrance (more dependence on genetic background) of the targeted gene. 2) Since controls are not a requisite, establishing a congenic strain is not necessary. The all-in-one model can be crossed with many strains to allow the study of Lrba on a wide genetic background, which is required for mimicking genetic diversity in the human population. F1 mice can be used directly for experiments. This can significantly reduce both time and cost. 3) The single mouse concept is also very useful in the study of gene-gene interactions. If a significant difference is detected in phenotypes from two mice, it may indicate the presence of a modifier gene(s) [41], which can be determined by the deep sequencing of the whole exome from the two mice [6]. Therefore, this model can be used to quickly discover gene-gene interactions, which are critical in the study of complex human diseases. 4) Knockout, overexpression, and reporter mouse models are usually generated separately, and only one model is studied in most labs due to time and cost. With the all-in-one model of the invention, LRBA expression can be manipulated in multiple ways and the three genotypes can be generated in a single mouse, making the data more comparable. 5) GWAS has failed to establish common variant risks for the majority of common diseases. The inventors' strategy, featuring sensitive phenotyping and rapid genetic background change, offers a quick and elegant tool for identifying the genes underlying common diseases in humans.

The preliminary data demonstrate that the phenotype of Lrba deficiency is highly dependent on the genetic background in mice. This supports the concept that LRBA regulates multiple critical immune regulators and further strengthens the necessity of generating an all-in-one mouse model. The data also demonstrates that this project is technically feasible.

1. Phenotype of Lrba KO depends on genetic background and LRBA regulates multiple critical immune genes. Lrba deficiency causes different phenotypes in different strains. As the amount of C57BL/6 background increases, so does the percentage of pups found positive for the Lrba knockout allele (FIG. 2A). LRBA is LPS-responsive and an anchoring protein for protein kinase A (PKA) [16,42]. LPS and PKA can activate NFκB, which is implicated in the pathogenesis of human immunodeficiency diseases [43]. This NFκB signaling may be affected by LRBA deficiency. The results show LRBA KD increases NFκB activity in a dose-dependent manner (FIG. 2B), increasing TNFα and IL10 (FIG. 2C) but downregulating CVID receptors (FIG. 2E). Like NFκB, MAPKs and AKT are critical downstream hubs of the TLR4/LPS signal transduction pathway [44]. LRBA KD downregulates ERK but upregulates p38, JNK (FIG. 2F) and AKT (FIG. 2G). Complementing, the preliminary data, Wech and Nagel reported decreased MAPK (ERK orthologue) and increased JNK upon rugose (LRBA orthologue) mutation [45].

2. Target vector and single guide RNA (gRNA)/Cas9 vector construction: The target vector was constructed from a BAC clone using homologous recombineering (HR). The subsequent engineering of the vector included multiple rounds of HR, and the CRISPR cloning method [19]. The correct cloning of the target vector was characterized by restriction digestion and confirmed by sequencing the TCC and the junctions between the 3′ arm/5′ arm/TCC/backbone regions. Portions of sequence traces around loxP sites show accurate cloning (FIGS. 3A-3C). A secretable luciferase gene was inserted in place of Lrba and used to show the tet-inducible system in the TCC function, which was as expected (FIG. 3D). The neo cassette in the target vector is not required by CRISPR and was removed by the plasmid pE-FLP resulting in a 5.9 kb TCC. The gRNA sequence was designed using the online CRISPR Design Tool and cloned into the pX330 vector [46]. Since the gRNA sequence spans the insertion site, the target vector does not have the sequence and will not be cleaved by the Cas9 nuclease. The gRNA sequence also has a SpeI site, which was used to identify gene mutations (FIG. 3E), in addition to a T7 Endonuclease I (T7EI) assay. Both assays indicated that the Lrba locus had been successfully mutated in the ES cells. The 7.4 kb insert was successfully knocked-in in ES cells (FIGS. 3A-3I) at high efficiency (˜70%). The long homologous arm (3, 9 kb) likely increased the targeting efficiency.

Since the phenotypes caused by the LRBA gene are highly dependent on genetic background and environmental factors, highly sensitive phenotyping and rapid genetic background change are essential in order to recapitulate LRBA deficient human disease in a mouse model. A unique mouse model that has these two prerequisites will be generated.

Generation an all-in-one mouse model in which Lrba expression can be turned on/off in a spatiotemporal and trackable manner.

The goal of this research was to generate the all-in-one Lrba mouse model by knocking in a transcription control cassette (TCC) into the Lrba locus in order to control Lrba expression in a spatiotemporal and trackable manner (FIGS. 4A-4C). The Cas9/gRNA system is currently used to quickly generate mouse models (˜3 months) at high efficiency (up to 78%) with ˜100% germline transmission [47-52] and low or no off-target mutation [53,54]. It causes DNA double strand break, which can be repaired by homology-directed-repair (HDR) pathways with a high efficiency that is 5000 times higher than that of traditional homologous recombination [55]. Due to these advantages, the one-step CRISPR/HDR generation of the mouse model technique will be used to insert the TCC into the Lrba genomic locus in order to expeditiously generate the all-in-one mouse model (FIG. 4B) [49,50].

Target Vector Design.

The target vector has been constructed (FIGS. 3A-3I). The CAG promoter in the original target vector will be removed by partial Cre recombination in bacteria [56] so that the TCC is reduced to ˜4 kb (FIGS. 4A-4C).The TCC contains 1) the Tet-On third generation system, which has significantly reduced background, is 100-fold more sensitive to Dox than the original Tet-On system and is widely used [57-59]; 2) FP reporter genes: iRFP670 RFP is a near-infrared fluorescent protein [60], and Aquamarine CFP is superior to the popular form ECFP [61]. Both genes were mouse-codon optimized. They are used to track Lrba promoter activity and Lrba expression by flow cytometry. 3) P2A peptide is used to link the two genes, allowing RFP and rtTA, or CFP and Lrba to be co-expressed by a single promoter [62]. P2A's small size (57 bp), its high self-cleavability, and its ability to produce equal molar ratio of the two proteins make it superior to IRES, which is large and causes differential expression of the two genes it links. 4) Splice sites are added to avoid ineffective translation of a CDS. The inventors have functionally tested the target vector in ES and H293 cells (FIGS. 3A-3I).

Generation of the All-in-One (a) Mice by CRISPR.

This will be done in collaboration as described [47]. In brief, T7 promoter will be added to the Cas9 coding sequence (CDS) and to Lrba gRNA by PCR [47]. The RNAs will be synthesized by T7 RNA polymerase and purified. Cas9 mRNA (100 ng/ml), gRNA (50 ng/ml), and a target plasmid DNA (200 ng/ml) will be injected into the fertilized B57BL/6 eggs. The genomic DNA from the targeted and the control mice, ranging from 8 to 12 days, will be extracted from clipped toes and used for PCR screening: The correct 5′ and 3′ end targeting will be confirmed by PCR protocol [10] by using the primers from the vector and the genomic DNA sequence outside of the short or the long arms (FIG. 3G). The PCR products are predicted to be 3.5 kb and 9.3 kb. The mice that are correctly targeted as identified by PCR will then be confirmed by Southern blot (FIG. 3I) [10]. For the 5′ targeting end, EcoRV-digested genomic DNA will be hybridized with a 5′ external probe with expected fragment sizes of 5 kb (wt) and 11 kb (targeted, T). The blot will then be stripped and hybridized with an rtTA internal probe with expected fragment sizes of 0 (wt) and 11 kb (T). Similarly, for the 3′ end, SfiI-digested genomic DNA will produce 25 kb (wt) and 15 kb (ko) bands using a 3′ external probe. The stripped blot will be hybridized with an internal probe with expected fragment sizes of 0 (wt) and 15 kb (T). Internal vector probes will then be used to detect incorrect insertions. Generation of αCreERT2 mice: As shown in FIG. 4B, the Cre gene which can be activated in the presence of tamoxifen (Tam) will be introduced into the α mice by mating with the ROSA26-creERT2 mice (JAX#008463) that ubiquitously express a Tam-inducible Cre recombinase (including B & T cells) [63]. This is more sensitive and specific to Tam than the ERT version [63]. The potential leaking of Tet-on [57-59] and CreERT2 [64] systems will be detect by real time PCR (FIG. 3H).

It is expected that the all-in-one mouse model will be successfully generated within one year. This model will be made available to scientists who are interested in LRBA. Potential pitfalls include: 1) The off-target mutations induced by CRISPR targeting is high in cultured cells [47,65], but is low or undetectable in mice [53,54]. The inventors will use the whole exon sequencing (WES) in order to detect any off-target mutations. If mutations are detected, the founders will be backcrossed with C57BL/6 mice multiple times until no mutations can be detected. 2) Although the size of the TCC (5.9 kb) was of concern in the previous application, the inventors have since successfully inserted the 7.4 kb (with neo) TCC into the Lrba locus in ES cells at ˜70% efficiency (FIGS. 3H-3I). Others have used the CRISPR to knockin 3 to ˜6 kb transgene [47,66]. No problems are expected with the insertion of the reduced TCC (˜4 kb) into the Lrba locus in mouse zygotes with the long homologous arms (3.5 and 9 kb), which will increase targeting efficiency [67]. If this targeting fails, the targeted ES clones (FIG. 3I) will be used to obtain this model.

Testing the functionality of the all-in-one mouse model in recapitulating the key features of LRBA deficient disease.

Accumulated evidence suggests that LRBA deficient disease results from the impaired regulation of cell death during lymphocyte development, as fewer mature lymphocytes but more immature lymphocytes were observed [11,12,17,18]. These lymphocytes, e.g. Treg cells [11] and class switched B cells [20,21], are significantly reduced (cytopenia), which may result in autoimmunity [11] and antibody deficiency, respectively [20,21]. Follicular lymphoid hyperplasia (ALPS) [12,20-22] and tissue infiltration of lymphocytes were also observed [21]. Both B and T lymphocytes undergo massive cell death at multiple developmental stages in order to eliminate non- or self-reactive lymphocytes. LRBA has two basic functions that can be involved in lymphocyte development. 1) Cell survival regulation. [13] LRBA knockdown increases cell survival in B cells (FIGS. 2A-2G), but LRBA deficiency increases apoptosis [20]. LRBA regulates multiple survival genes, including NF-κB which control the lymphocyte development [68]. The self-reactive B or T cells with increased cell survival may escape from cell death and then predispose LRBA deficient patients to autoimmunity. 2) Vesicle trafficking regulation,[16,69] which is required for homeostasis of membrane receptors [70]. LRBA regulates several cell membrane receptors [9,13,18,20] (FIGS. 2A-2G). It likely also regulates receptors that can induce cell death or survival of lymphocytes, such as BCR and TCR. Thus, LRBA likely plays an important role in lymphocyte development, deregulation of which is the major cause of autoimmunity and immunodeficiency. These key features, including Lrba's role on cell survival at multiple developmental stages of lymphocytes, will be examined in αCreERT2 and wt control mice with or without TAM treatment on two genetic backgrounds by flow cytometry*.

The phenotype of αCreERT2 mice generated can be studied before adding Dox (Lrba negative), after adding Dox (Lrba positive), or after Dox withdrawal (Lrba negative) in the same mouse. Furthermore, mosaic lymphocytes with the three genotypes (wt, het, and ko) can be generated by incomplete Cre recombination in the same mouse (FIGS. 4A-4C). (a) Dox-induced Lrba gene expression. The αCreERT2 and wt C57BL/6 (used as Dox treatment control) male mice of 6 wks age will be treated with or without Dox. Dox will activate Lrba expression in the αCreERT2 mice. (b) Tam-induced Cre mosaic recombination. Three genotypes will be produced in the same αCreERT2 mouse by incomplete Cre mosaic recombination (FIG. 1A and FIG. 4B). These genotypes can then be distinguished by FPs (FIG. 4B): Lrba ko, red; Lrba het, red and cyan; Lrba wt, cyan. The conditions (dosing and time) of Tam treatment will be optimized to obtain roughly equal numbers of the three types of lymphocytes, as determined by flow cytometry and real-time PCR (FIG. 3H). The αCreERT2 mice will receive 0, 3, or 9 mg of Tam (ip injection) for 1 to 5 consecutive days. On day 24, the mice will be treated with Tam (i.p.) at the optimized condition to induce partial Cre recombination, which will produce the three types of lymphocytes [71]. On days 0, 10, 17, and 31, blood will be collected from the submandibular vein and subjected to flow cytometry (FIG. 5). On day 45, mice will be euthanized. The bone marrow, spleen, thymus, whole blood, lymph nodes, lung and intestine will be collected for flow cytometry or tissue sectioning for fluorescent microscopy [72].

Tesing whether Lrba expression can be turned on/off in a spatiotemporal and trackable manner.

This test is informative as it will allow phenotype to be attributed specifically to Lrba if they change in response to Lrba expression being switched on/off. As mouse peripheral blood is limited, the inventors will use a no-lyse, no-wash staining flow cytometry technique that uses 20 μL of whole blood for each analysis [73,74]. The expression of FPs, two CVID receptors:TACI and BAFFR, critical regulators of cell survival [75,76] in the peripheral white blood cells will be examined by flow cytometry.

Recapitulation of LRBA deficiency on the development of lymphocytes.

In addition, LRBA is expressed ubiquitously, especially in hematopoietic cells and stem cells [13,15]. The inventors will use the multiparametric flow cytometry developed by BD Biosciences to study the development of B and T lymphocytes in this model. (a) Analysis of B-cell developmental stages in mouse bone marrow. [77] Seven different developmental phases can be discriminated in bone marrow by a panel of seven B cell surface markers. Pre-pro-B, Pro-B, and Pre-B cells can be distinguished within the low positive CD45R population based on their differential expression of BP1 and CD24. Immature, transitional, and early and late mature B cells could be segregated based on differential expression of IgM and IgD [77]. To study the effect of Lrba ko in the periphery, mature B cell presence in mouse spleens will also be determined. (b) Analysis of T-cell developmental stages in thymus. [78] The six developmental stages, four double-negative (DN1, DN2, DN3, DN4), double-positive (DP) and single-positive (SP), can be discriminated in thymus by an 8-color panel of cell surface markers. B220 negative events will be gated first, and then CD4 vs CD8 will identify the DN, DP, and SP cell populations. CD44 vs CD25 will identify the four substages (DN1 to DN4) in DN cell population. The low, intermediate, and high expression levels of TCR β corresponds to DN, DP and SP cells. CD69 and CD5 will be included in the panel because they are indicators for positive selection and the intensity of TCRs and self MHC-peptides interactions. Mature T cell presence in mouse spleens and lymph nodes will also be determined to study the effect of Lrba ko in the periphery. The activation state of B cells or T cells will also be determined in the periphery using MHC class II, CD40 and CD86 for B cells and, CD44, CD62L and CD25 for T cells, respectively. *Flow cytometry methodologies: LSR-II flow cytometer, which has an analysis rate of up to 40,000 cells per second, and the capacity to measure 15 cell markers. 40 samples can be analyzed within a couple of hours. 1) To exclude cell aggregates, two sequential gates of scatter width vs height signals will be applied. The singlet population will then be gated by forward scatter vs side scatter to exclude dead cells and debris. “Live cells” will be gated using a contour plot and then switch to a dot plot for easy monitoring of acquisition. 2) A FMO-control (Fluorescence Minus One) is a control sample composed of all antibody labels except one, and will be used as a negative control in place of an isotype control for that antibody staining. 3) All antibodies will be titrated. 4) The fluorophores used by BD that overlap with iRFP of CFP will be replaced with other fluorophores. (c) Others: 1) The One Step Staining Mouse Treg Flow™ Kit (BioLegend) will be used to detect Treg cells in mouse spleens. 2) Plasma antibody isotyping (IgG1, IgG2a, IgG2b, IgG3, IgA and IgM) will be performed using the Pierce Rapid ELISA Mouse mAb Isotyping Kit. 3) Lymph nodes, lung and intestine sections will be analyzed by fluorescent microscopy [72] to examine lymphoproliferation and lymphocyte infiltration. 4) Comprehensive standardized gross and histopathologic analyses will be performed, including the analyses of organ weights, serum chemistries and hematology. The inventors will use 12 age-matched mice of both sexes, 6 mutant and 6 wild-type controls for the analyses.

Recapitulation of LRBA deficiency on different genetic backgrounds: The phenotype is different in the two strains (FIG. 2A). The αCreERT2 mice will be mated with 129P2 mice, and then the same set of experiments described above will be carried out. Results from the two genetic backgrounds will then be compared to find background-specific phenotypes (FIGS. 1A-1B) to identify modifiers.

The impact of LRBA deficiency on the development of B or T cells at each stage has not been investigated yet. The all-in-one mouse model will allow highly sensitive detection of any abnormalities in B and T cell development caused by Lrba deficiency, which may not be detectable with the current techniques. 1) Based on human data, it is expected, as shown in FIG. 4C, that the percentage of immature lymphocytes will follow the order of Ko>Het>Wt, at each developmental stage, while the percentage of the mature cells including Treg will have the reversed order of Ko<Het<Wt. However, this prediction is based on the intrinsic effects of Lrba. It is possible that different types of cells can affect each other by trans-effects. To distinguish cell intrinsic-effects from trans-effects, the same experiments in will be carried out but with variable starting percentages of the three types of cells by treating the αCreERT2 mice with the TAM-treating conditions as determined above. The correlation coefficients of the results will be calculated to determine if there are any trans-effects [79]. 2) It is assumed that the Cre recombination efficiency is similar in the bone marrow and in the periphery. To increase accuracy, the inventors will detect the Cre recombination efficiency in the bone marrow to establish a correlation between the TAM treatment condition and the recombination efficiency. 3) The inventors expect some phenotypes are different, others are the same on the two backgrounds. The new phenotypes can be isolated and stabilized by further backcrossing, then WGS will be used to identify modifiers. 4) It is expected that the FP expression will respond to Dox as shown in FIG. 1A. After Tam treatment, it is expected that the three genotypes, i.e. wt, het, and ko can be identified in B cells in the same αCreERT2. 5) Plasma antibodies of Lrba deficient mice are expected to be lower than that of wt mice. 6) lymphoproliferation and lymphocyte infiltration may be observed in lymph nodes, lung and intestine sections.

### Supplemental Disclosure 2

The animals and cells obtained therefrom are an elegant tool to better recapitulate complex human diseases. In one embodiment, the invention provides a conditional gene knockout technique to study genes in a single animal at high resolution. Currently, clear and consistent phenotypes are the exception rather than the rule. This is because the variations between individuals can mask a gene's contribution to a phenotype. The invention can eliminate these variations by allowing the study of three genotypes of a gene in a single animal in a single flow cytometry tube. Phenotyping sensitivity thus can be greatly increased, e.g., by up to one million times theoretically, and the technique can detect the phenotypes undetectable currently.

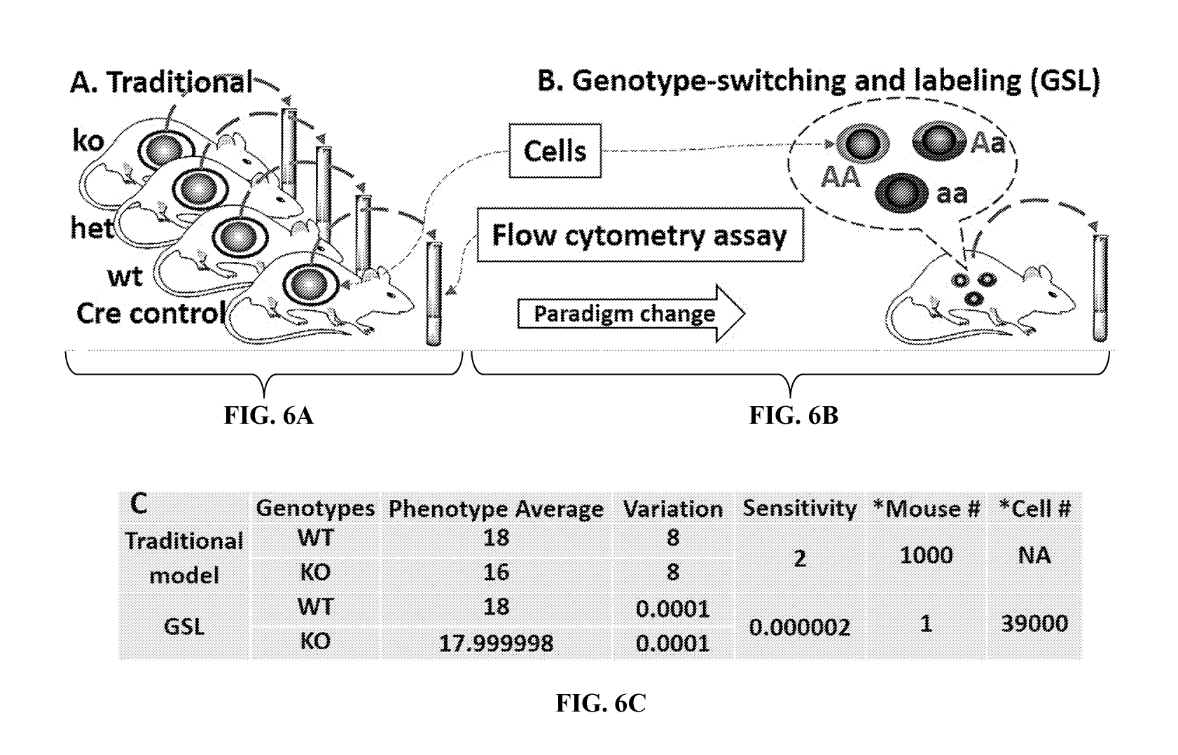

Optionally, the targeting strategy can be modified to reduce the transgene size and simplify this animal model while retaining the advantages of the conditional knockout technique. The present invention provides a conditional gene knockout technique to study the genotype-phenotype relationship (GPr) at high resolution (FIGS. 6A-C), which is critical to discovering the etiologies of complex human diseases. A clear GPr is lacking in the patients with the mutation of the lipopolysaccharide-responsive beige-like anchor (LRBA)2,3 as the clinical manifestations are highly variable [83,84]. The poor GPr is because the variations between subjects can mask a gene's contribution to a phenotype [85]. An impractically large sample size, e.g., 500,000 subjects, is often required to obtain statistically significant data [86]. To eliminate the variations, the inventors provide and label the three genotypes, wild type (wt), heterozygote (het), and knockout (ko), of a gene with fluorescent proteins (FP) in a single mouse (FIG. 6A). The three genotypes of large numbers of cells, e.g., ˜2×106 T regulatory cells, from a single animal thus can be analyzed in a single tube by flow cytometry. The variations between these cells can be eliminated, because these cells are from the same animal, and thus have the same genetic background, epigenetic modifications, and environmental inputs. With large sample size and eliminated variations, the phenotyping sensitivity can be greatly increased, e.g., by one million times theoretically (FIG. 6C). This technique (GSL) thus can detect phenotypes that cannot be detected currently. Furthermore, it can replace the current method to obtain data with much higher resolution in most cases where the two methods are doing the same jobs except that “the genotypes are labeled on cells or on tubes,” and that the cells are from one animal (e.g., mouse) or multiple animals (e.g., mice), respectively. As a proof of principle, the inventors will use the clustered regularly interspaced short palindromic repeats (CRISPRs) technologies to conditionally knockout Lrba in mouse embryonic stem (ES) cells to test this concept. The inventors have done the pioneering work on LRBA [81,82], and generated a conditional knockout model [81,87]. The inventors have also developed two novel CRISPR techniques to facilitate gene targeting [88,89].

Targeting Vector Construction.

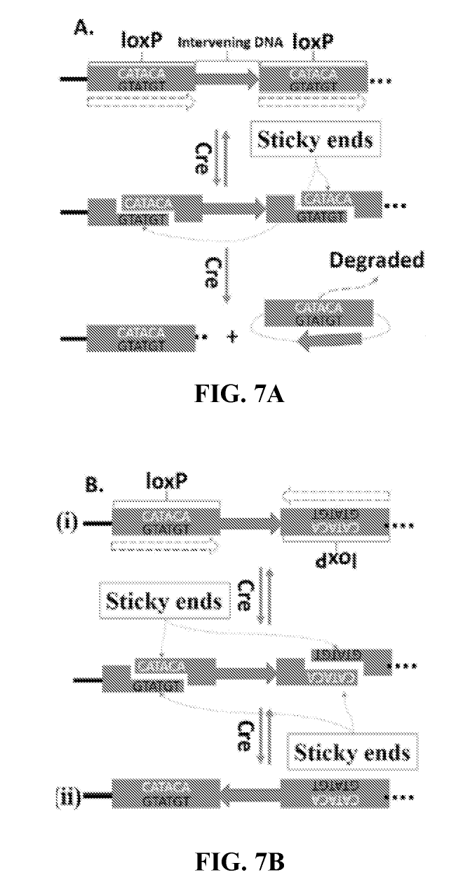

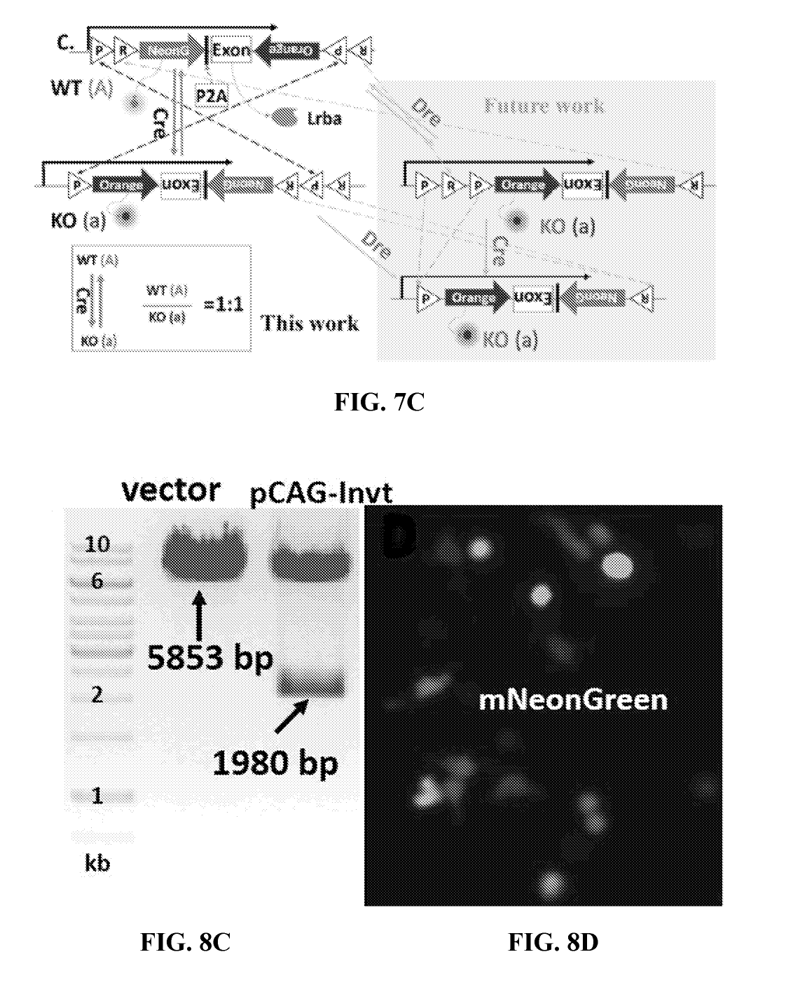

(1) Redesigned targeting vector: Fluorescent proteins (FP) [92] and site-specific recombinases, e.g., Cre, are indispensable and widely-used tools in the analysis of gene functions in a visible and spatiotemporal manner, respectively [93]. There are two potential disadvantages associated with the inventors' former model. One is that it may be difficult to control the Cre-recombination to obtain equal numbers of wt and ko alleles. Another is that the default inactivation of the targeted gene by the knockin can be lethal if this gene is essential for survival. To overcome these potential issues, the inventors redesigned kn transgene (GSLC, ˜2 kb. FIG. 7C) with the following improvements: a) Cre-mediated recombination will produce equal numbers of wt and ko alleles (FIG. 7C, this work part), and b) unlike the inventors' former strategy but like current conditional knockout technique, the new strategy will not inactivate the targeted gene by the kn transgene, and can produce the three genotypes in separate animals to study the extrinsic function of the targeted gene (FIG. 7C, future work part). The inventors' former strategy does not have such function [94].

(2) Innovation: While this GSL model can be used as a regular conditional knockout model, it will allow investigators to generate and respectively label the three genotypes of the targeted gene to study the phenotypes of a gene in a single (e.g., a single mouse) with high resolution (FIG. 1). Therefore, the GSL model will represent a significant improvement to the current conditional knockout technique. Simultaneously, gene knockout and labeling by using Cre-mediated inversion/excision have been widely used, but thus far they can only label the mutated allele and thus cannot distinguish the three genotypes [97]. The mosaic analysis with double markers (MADM) system can produce three genotypes with distinct FPs in a mouse. However, its labeling efficiency is too low (5%, at most), while Brainbow only labels cells. In contrast, GSL technique can be used to label both genotypes and cells with ˜100% efficiency.

(3) Cloning strategy of the redesigned targeting vector: The GSLC that has polycloning sites at the both ends has been cloned (FIG. 6C). Two Lrba homologous arms (˜1 kb each) will be PCR amplified and cloned into the polycloning sites of the above vector as described [88,89].

Obtaining Knock-In ES Cell Clones.

The Cas9/gRNA system causes DNA double strand break, which can be repaired by the homology-directed-repair (HDR) pathway.26 Using this method, the inventors successfully inserted a 7.4 kb fragment into ES cells at high efficiency [89]. The above resultant targeting vector will be electroporated into ES cells. The NeonGreen (like GFP)-positive clones will be picked up and subjected to 3′ and 5′ end PCR screening of homologous recombination [89]. The correctly targeted clones will be confirmed by Southern blot [87,89].

Testing the Functionality of the GSLC.

GSLC is used to switch and label the genotypes, and will be tested in cell culture prior to generating a mouse model, which is costly and time-consuming.

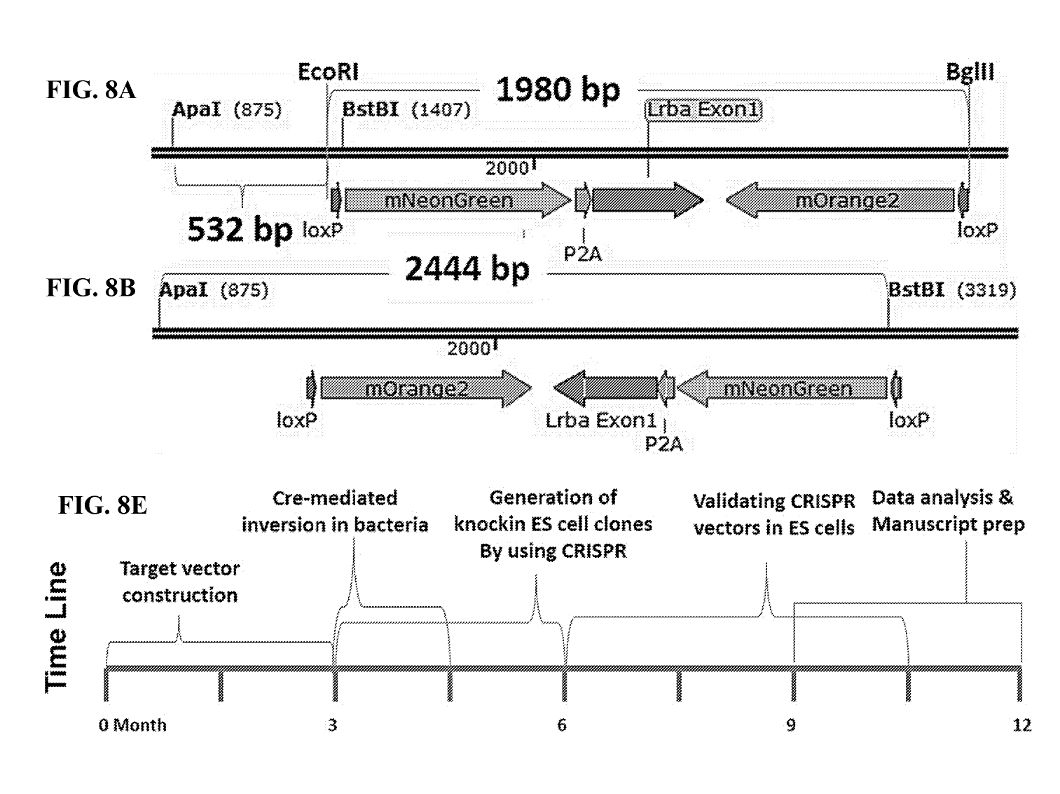

(1) The Cre recombinasemediated inversion will be tested in bacteria. Cre recombinase-mediated inversion will be examined by transforming the 706-Cre plasmid (Gene Bridges GmbH) into the bacteria containing the targeting vector, and then by restriction enzyme digestion of the isolated DNA (FIGS. 8A and 8B) following the manufacturer's protocol. The presence of a 2.4 kb band in a DNA gel will indicate successful inversion. The ratios of the inverted and original plasmids can be estimated by the intensity of the two bands (0.5 & 2.4 kb), or by re-transforming the mixture of the two plasmids and counting their colonies. It is expected that the numbers of the colonies that harbor the two plasmids are equal, indicating that the rates of Cre-mediated reactions at both directions are equal.

(2) The functionality of the GSLC will be tested in ES cells. The GSLC will be knocked into the Lrba genomic locus in ES cells as previously [89]. Green FP positive clones will be picked up and screened by PCR, and confirmed by Southern blot following the inventors' methods [89]. The correctly targeted ES clones will be infected with the recombinant retroviruses (MSCV.CreERT2.puro. Addgene) and selected by puromycin for stable clones, which will be used for Tamoxifen (TAM)-induced Cre-cleavage assay. The exclusive expression of Neongreen and Orange2 will be directly detected by flow cytometry. The association of the genotypes with the FP colors will also be confirmed by sorting the three genotypes of cells and conducting PCR and Western blot, and further confirmed by Southern blot [89]. It is expected that the three genotypes of Lrba will be generated and specifically labeled in ES cells with the ratios: wt:het:ko=1:2:1. The knockin vector may need to be modified to obtain optimal genotype-switching and labeling results. This can be easily done by using the CRISPR cloning technique [88]. In summary, this technique can be used to obtain high resolution data while retaining all the function of the current conditional knockout technique. This is a significant improvement.

### REFERENCES FOR SUPPLEMENTAL DISCLOSURES 1 and 2

1. Pearson, H. Surviving a knockout blow. Nature 415, 8-9 (2002).
2. Manolio, T. A. & Collins, F. S. Genes, environment, health, and disease: facing up to complexity. Hum Hered 63, 63-6 (2007).
3. Keane, T. M. et al. Mouse genomic variation and its effect on phenotypes and gene regulation. Nature 477, 289-94 (2011).
4. Belizario, J. E. The humankind genome: from genetic diversity to the origin of human diseases. Genome 56, 705-16 (2013).
5. Cutting, G. R. Modifier genes in Mendelian disorders: the example of cystic fibrosis. Ann NY Acad Sci 1214, 57-69 (2010).
6. Bull, K. R. et al. Unlocking the bottleneck in forward genetics using whole-genome sequencing and identity by descent to isolate causative mutations. PLoS Genet 9, e1003219 (2013).
7. Zong, H., Espinosa, J. S., Su, H. H., Muzumdar, M. D. & Luo, L. Mosaic analysis with double markers in mice. Cell 121, 479-92 (2005).
8. Tanaka, K. F. et al. Flexible Accelerated STOP Tetracycline Operator-knockin (FAST): a versatile and efficient new gene modulating system. Biol Psychiatry 67, 770-3 (2010).
9. Volders, K. et al. Drosophila rugose is a functional homolog of mammalian Neurobeachin and affects synaptic architecture, brain morphology, and associative learning. J Neurosci 32, 15193-204 (2012).
10. Wang, J. W. et al. Influence of SHIP on the NK repertoire and allogeneic bone marrow transplantation. Science 295, 2094-7 (2002).
11. Charbonnier, L. M. et al. Regulatory T-cell deficiency and immune dysregulation, polyendocrinopathy, enteropathy, X-linked-like disorder caused by loss-of-function mutations in LRBA. J Allergy Clin Immunol 135, 217-227 e9 (2015).
12. Seidel, M. G. et al. Long-term remission after allogeneic hematopoietic stem cell transplantation in LPS-responsive beige-like anchor (LRBA) deficiency. J Allergy Clin Immunol (2014).
13. Wang, J. W. et al. Deregulated expression of LRBA facilitates cancer cell growth. Oncogene 23, 4089-97 (2004).
14. Andres, S. A., Brock, G. N. & Wittliff, J. L. Interrogating differences in expression of targeted gene sets to predict breast cancer outcome. BMC Cancer 13, 326 (2013).
15. Lopez-Herrera, G. et al. LRBA Deficiency In Mexican Patients With Common Variable Immunodeficiency. The 16th Biennial Meeting of the European Society for Immunodeficiencies (ESID 2014) (2014).
16. Wang, J. W., Howson, J., Haller, E. & Kerr, W. G. Identification of a novel lipopolysaccharide-inducible gene with key features of both A kinase anchor proteins and chsl/beige proteins. J Immunol 166, 4586-95 (2001).
17. Shamloula, H. K. et al. rugose (rg), a Drosophila A kinase anchor protein, is required for retinal pattern formation and interacts genetically with multiple signaling pathways. Genetics 161, 693-710 (2002).
18. Yatim, A. et al. NOTCH1 nuclear interactome reveals key regulators of its transcriptional activity and oncogenic function. Mol Cell 48, 445-58 (2012).
19. Wang, J.-W., Wang, A., Li, K., Jin, S. & Lockey, R. F. CRISPR/Cas9 nuclease combined with Gibson assembly for seamless cloning into large vectors. Biotechniques. In press.(2015).
20. Lopez-Herrera, G. et al. Deleterious mutations in LRBA are associated with a syndrome of immune deficiency and autoimmunity. Am J Hum Genet 90, 986-1001 (2012).
21. Alangari, A. et al. LPS-responsive beige-like anchor (LRBA) gene mutation in a family with inflammatory bowel disease and combined immunodeficiency. J Allergy Clin Immunol 130, 481-8 e2 (2012).
22. Burns, S. O. et al. LRBA gene deletion in a patient presenting with autoimmunity without hypogammaglobulinemia. J Allergy Clin Immunol (2012).
23. Serwas, N. K. et al. Atypical Manifestation of LRBA Deficiency with Predominant IBD-like Phenotype. Inflamm Bowel Dis 21, 40-7 (2015).
24. Gamez-Diaz, L. et al. NEW DEFICIENCY CAUSING EARLY-ONSET CVID: HOW TO DIAGNOSE LRBA DEFECTS? J Clin Immunol 32, 77-77 (2012).
25. Ridgway, W. M., Healy, B., Smink, L. J., Rainbow, D. & Wicker, L. S. New tools for defining the ‘genetic background’ of inbred mouse strains. Nat Immunol 8, 669-73 (2007).
26. Ridgway, W. M. A new tool for dissecting genetic control of type 1 diabetes. Diabetes 63, 56-8 (2014).
27. Eisener-Dorman, A. F., Lawrence, D. A. & Bolivar, V. J. Cautionary insights on knockout mouse studies: the gene or not the gene? Brain Behav Immun 23, 318-24 (2009).
28. Sundberg, J. P., Roopenian, D. C., Liu, E. T. & Schofield, P. N. The Cinderella effect: searching for the best fit between mouse models and human diseases. J Invest Dermatol 133, 2509-13 (2013).
29. Kalliokoski, O., Teilmann, A. C., Jacobsen, K. R., Abelson, K. S. & Hau, J. The Lonely Mouse—Single Housing Affects Serotonergic Signaling Integrity Measured by 8-OH-DPAT-Induced Hypothermia in Male Mice. PLoS One 9, e111065 (2014).
30. Ermann, J. & Glimcher, L. H. After GWAS: mice to the rescue? Curr Opin Immunol 24, 564-70 (2012).
31. Kado, S. et al. Intestinal microflora are necessary for development of spontaneous adenocarcinoma of the large intestine in T-cell receptor beta chain and p53 double-knockout mice. Cancer Res 61, 2395-8 (2001).
32. Franklin, C. L. Microbial considerations in genetically engineered mouse research. ILAR J 47, 141-55 (2006).
33. Treuting, P. M., Clifford, C. B., Sellers, R. S. & Brayton, C. F. Of mice and microflora: considerations for genetically engineered mice. Vet Pathol 49, 44-63 (2012).
34. Watkins-Chow, D. E. & Pavan, W. J. Genomic copy number and expression variation within the C57BL/6J inbred mouse strain. Genome Res 18, 60-6 (2008).
35. Bryant, C. D. et al. Behavioral differences among C57BL/6 substrains: implications for transgenic and knockout studies. J Neurogenet 22, 315-31 (2008).
36. Saeed, S. et al. Epigenetic programming of monocyte-to-macrophage differentiation and trained innate immunity. Science 345, 1251086 (2014).
37. Stevens, J. C., Banks, G. T., Festing, M. F. & Fisher, E. M. Quiet mutations in inbred strains of mice. Trends Mol Med 13, 512-9 (2007).
38. Vogel, G. Genetics. Scientists dream of 1001 complex mice. Science 301, 456-7 (2003).
39. Seok, J. et al. Genomic responses in mouse models poorly mimic human inflammatory diseases. Proc Natl Acad Sci USA 110, 3507-12 (2013).
40. Rivera, J. & Tessarollo, L. Genetic background and the dilemma of translating mouse studies to humans. Immunity 28, 1-4 (2008).
41. Dietrich, W. F. et al. Genetic identification of Mom-1, a major modifier locus affecting Min-induced intestinal neoplasia in the mouse. Cell 75, 631-9 (1993).
42. Kerr, W. G., Heller, M. & Herzenberg, L. A. Analysis of lipopolysaccharide-response genes in B-lineage cells demonstrates that they can have differentiation stage-restricted expression and contain SH2 domains. Proc Natl Acad Sci USA 93, 3947-52 (1996).
43. Kurylowicz, A. & Nauman, J. The role of nuclear factor-kappaB in the development of autoimmune diseases: a link between genes and environment. Acta Biochim Pol 55, 629-47 (2008).
44. Oeckinghaus, A., Hayden, M. S. & Ghosh, S. Crosstalk in NF-kappaB signaling pathways. Nat Immunol 12, 695-708 (2011).
45. Wech, I. & Nagel, A. C. Mutations in rugose promote cell type-specific apoptosis in the Drosophila eye. Cell Death Differ 12, 145-52 (2005).
46. Cong, L. et al. Multiplex genome engineering using CRISPR/Cas systems. Science 339, 819-23.
47. Yang, H. et al. One-step generation of mice carrying reporter and conditional alleles by CRISPR/Cas-mediated genome engineering. Cell 154, 1370-9 (2013).
48. Zhou, J. et al. Dual sgRNAs facilitate CRISPR/Cas9-mediated mouse genome targeting. FEBS J 281, 1717-25 (2014).
49. Wang, H. et al. One-step generation of mice carrying mutations in multiple genes by CRISPR/Cas-mediated genome engineering. Cell 153, 910-8.
50. Yang, H. et al. One-step generation of mice carrying reporter and conditional alleles by CRISPR/Cas-mediated genome engineering. Cell 154, 1370-9.
51. Fujii, W., Kawasaki, K., Sugiura, K. & Naito, K. Efficient generation of large-scale genome-modified mice using gRNA and CAS9 endonuclease. Nucleic Acids Res 41, e187.
52. Jinek, M. et al. A programmable dual-RNA-guided DNA endonuclease in adaptive bacterial immunity. Science 337, 816-21 (2012).
53. Inui, M. et al. Rapid generation of mouse models with defined point mutations by the CRISPR/Cas9 system. Sci Rep 4, 5396 (2014).
54. Mashiko, D. et al. Generation of mutant mice by pronuclear injection of circular plasmid expressing Cas9 and single guided RNA. Sci Rep 3, 3355 (2013).
55. Donoho, G., Jasin, M. & Berg, P. Analysis of gene targeting and intrachromosomal homologous recombination stimulated by genomic double-strand breaks in mouse embryonic stem cells. Mol Cell Biol 18, 4070-8 (1998).
56. Warming, S., Costantino, N., Court, D. L., Jenkins, N. A. & Copeland, N. G. Simple and highly efficient BAC recombineering using galK selection. Nucleic Acids Res 33, e36 (2005).
57. Zhou, X., Vink, M., Klaver, B., Berkhout, B. & Das, A. T. Optimization of the Tet-On system for regulated gene expression through viral evolution. Gene Ther 13, 1382-90 (2006).
58. Loew, R., Heinz, N., Hampf, M., Bujard, H. & Gossen, M. Improved Tet-responsive promoters with minimized background expression. BMC Biotechnol 10, 81 (2010).
59. Kistner, A. et al. Doxycycline-mediated quantitative and tissue-specific control of gene expression in transgenic mice. Proc Natl Acad Sci USA 93, 10933-8 (1996).
60. Shcherbakova, D. M. & Verkhusha, V. V. Near-infrared fluorescent proteins for multicolor in vivo imaging. Nat Methods 10, 751-4 (2013).
61. Erard, M. et al. Minimum set of mutations needed to optimize cyan fluorescent proteins for live cell imaging. Mol Biosyst 9, 258-67 (2013).
62. Kim, J. H. et al. High cleavage efficiency of a 2A peptide derived from porcine teschovirus-1 in human cell lines, zebrafish and mice. PLoS One 6, e18556 (2011).
63. Ventura, A. et al. Restoration of p53 function leads to tumour regression in vivo. Nature 445, 661-5 (2007).
64. Madisen, L. et al. A robust and high-throughput Cre reporting and characterization system for the whole mouse brain. Nat Neurosci 13, 133-40 (2010).
65. Fu, Y. et al. High-frequency off-target mutagenesis induced by CRISPR-Cas nucleases in human cells. Nat Biotechnol 31, 822-6 (2013).
66. Kimura, Y., Hisano, Y., Kawahara, A. & Higashijima, S. Efficient generation of knock-in transgenic zebrafish carrying reporter/driver genes by CRISPR/Cas9-mediated genome engineering. Sci Rep 4, 6545 (2014).
67. Li, K., Wang, G., Andersen, T., Zhou, P. & Pu, W. T. Optimization of genome engineering approaches with the CRISPR/Cas9 system. PLoS One 9, e105779 (2014).
68. Gerondakis, S., Fulford, T. S., Messina, N. L. & Grumont, R. J. NF-kappaB control of T cell development. Nat Immunol 15, 15-25 (2014).
69. Reiser, M. A., Li, K., Lockey, R. F. & Wang, J. W. Lipopolysaccharide Responsive Beige-Like Anchor Subcellular Localization Involving in Vesicle Trafficking Responsive to Lipopolysaccharide. Austin Journal of Clinical Immunology 1, 8 (2014).
70. Wiley, H. S. Trafficking of the ErbB receptors and its influence on signaling. Exp Cell Res 284, 78-88. (2003).
71. Hayashi, S. & McMahon, A. P. Efficient recombination in diverse tissues by a tamoxifen-inducible form of Cre: a tool for temporally regulated gene activation/inactivation in the mouse. Dev Biol 244, 305-18 (2002).
72. Cai, D., Cohen, K. B., Luo, T., Lichtman, J. W. & Sanes, J. R. Improved tools for the Brainbow toolbox. Nat Methods 10, 540-7 (2013).
73. Weaver, J. L., Broud, D. D., McKinnon, K. & Germolec, D. R. Serial phenotypic analysis of mouse peripheral blood leukocytes. Toxicol Mech Methods 12, 95-118 (2002).
74. Weaver, J. L., McKinnon, K. & Germolec, D. R. Phenotypic analysis using very small volumes of blood. Curr Protoc Cytom Chapter 6, Unit 6 30 (2010).
75. Shulga-Morskaya, S. et al. B cell-activating factor belonging to the TNF family acts through separate receptors to support B cell survival and T cell-independent antibody formation. J Immunol 173, 2331-41 (2004).
76. Seshasayee, D. et al. Loss of TACI causes fatal lymphoproliferation and autoimmunity, establishing TACI as an inhibitory BLyS receptor. Immunity 18, 279-88 (2003).
77. BDbiosciences. B-Cell Research, Flow cytometry tools for the study of B-cell biology https://www.bdbiosciences.com/documents/Bcell_Brochure.pdf.
78. BDBiosciences. Studying Mouse Thymocyte Development using Multiparametric Flow Cytometry: An Efficient Method to Improve an 8-Color Panel on the BD FACSVerse™ System. BD Biosciences Application Note, 1-15 (2012).
79. Harrison, D. E. Competitive repopulation: a new assay for long-term stem cell functional capacity. Blood 55, 77-81 (1980).
80. Pearson, H. Surviving a knockout blow. Nature 415, 8-9 (2002).
81. Wang, J. W., Howson, J., Haller, E. & Kerr, W. G. Identification of a novel lipopolysaccharide-inducible gene with key features of both A kinase anchor proteins and chsl/beige proteins. J Immunol 166, 4586-95 (2001).
82. Wang, J. W., Gamsby, J. J., Highfill, S. L., Mora, L. B., Bloom, G. C., Yeatman, T. J., Pan, T. C., Ramne, A. L., Chodosh, L. A., Cress, W. D., Chen, J. & Kerr, W. G. Deregulated expression of LRBA facilitates cancer cell growth. Oncogene 23, 4089-97 (2004).
83. Seidel, M. G., Hirschmugl, T., Gamez-Diaz, L., Schwinger, W., Serwas, N., Deutschmann, A., Gorkiewicz, G., Zenz, W., Windpassinger, C., Grimbacher, B., Urban, C. & Bortug, K. Long-term remission after allogeneic hematopoietic stem cell transplantation in LPS-responsive beige-like anchor (LRBA) deficiency. J Allergy Clin Immunol (2014).
84. Alangari, A., Alsultan, A., Adly, N., Massaad, M. J., Kiani, I. S., Aljebreen, A., Raddaoui, E., Almomen, A. K., Al-Muhsen, S., Geha, R. S. & Alkuraya, F. S. LPS-responsive beige-like anchor (LRBA) gene mutation in a family with inflammatory bowel disease and combined immunodeficiency. J Allergy Clin Immunol 130, 481-8 e2 (2012).
85. Eisener-Dorman, A. F., Lawrence, D. A. & Bolivar, V. J. Cautionary insights on knockout mouse studies: the gene or not the gene? Brain Behav Immun 23, 318-24 (2009).
86. Zuk, O., Hechter, E., Sunyaev, S. R. & Lander, E. S. The mystery of missing heritability: Genetic interactions create phantom heritability. Proc Natl Acad Sci USA 109, 1193-8 (2012).
87. Wang, J. W., Howson, J. M., Ghansah, T., Desponts, C., Ninos, J. M., May, S. L., Nguyen, K. H., Toyama-Sorimachi, N. & Kerr, W. G. Influence of SHIP on the NK repertoire and allogeneic bone marrow transplantation. Science 295, 2094-7 (2002).
88. Wang, J. W., Wang, A., Li, K., Wang, B., Jin, S., Reiser, M. & Lockey, R. F. CRISPR/Cas9 nuclease cleavage combined with Gibson assembly for seamless cloning. BioTechniques 58, 161-70 (2015).
89. Wang, B., Li, K., Wang, A., Reiser, M., Saunders, T. L., Lockey, R. F. & Wang, J. W. Highly efficient CRISPR/HDR-mediated knockin in mouse embryonic stem cells and zygotes. BioTechniques. 59, 201-208 (2015).
90. Boston-University-and-Boston-Medical-Center. Sample Size Calculations. www.bu.edu website.
91. Gamez-Diaz, L., August, D., Stepensky, P., Revel-Vilk, S., Seidel, M. G., Noriko, M., Morio, T., Worth, A. J., Blessing, J., Van de Veerdonk, F., Feuchtinger, T., Kanariou, M., Schmitt-Graeff, A., Jung, S., Seneviratne, S., Burns, S., Belohradsky, B. H., Rezaei, N., Bakhtiar, S., Speckmann, C., Jordan, M. & Grimbacher, B. The extended phenotype of LPS-responsive beige-like anchor protein (LRBA) deficiency. J Allergy Clin Immunol 137, 223-30 (2016).
92. Chudakov, D. M., Matz, M. V., Lukyanov, S. & Lukyanov, K. A. Fluorescent proteins and their applications in imaging living cells and tissues. Physiol Rev 90, 1103-63 (2010).
93. Branda, C. S. & Dymecki, S. M. Talking about a revolution: The impact of site-specific recombinases on genetic analyses in mice. Dev Cell 6, 7-28 (2004).
94. Anastassiadis, K., Fu, J., Patsch, C., Hu, S., Weidlich, S., Duerschke, K., Buchholz, F., Edenhofer, F. & Stewart, A. F. Dre recombinase, like Cre, is a highly efficient site-specific recombinase in E. coli, mammalian cells and mice. Dis Model Mech 2, 508-15 (2009).
95. Wan, Y. Y. & Flavell, R. A. Identifying Foxp3-expressing suppressor T cells with a bicistronic reporter. Proc Natl Acad Sci USA 102, 5126-31 (2005).
96. Dean, K. M. & Palmer, A. E. Advances in fluorescence labeling strategies for dynamic cellular imaging. Nat Chem Biol 10, 512-23 (2014).
97. Jungke, P., Hans, S., Gupta, M., Machate, A., Zoller, D. & Brand, M. Generation of a conditional limala allele in zebrafish using the FLEx switch technology. Genesis 54, 19-28 (2016).

### Exemplified Embodiments

Examples of embodiments of the invention include, but are not limited to:

Embodiment 1: A non-human animal model comprising three populations of a cell type, wherein each cell population has a different genotype for a gene, wherein the three cell populations comprise:

(a) a first population of cells having a wild-type gene;

(b) a second population of cells heterozygous for the gene; and

(c) a third population of cells having an inactivated version of the gene;

wherein the three populations of cells include a transcription control cassette operably linked at the genomic locus of the gene.

Embodiment 2: The non-human animal model of embodiment 1, wherein the three populations of cells comprise a first detectable label, a second detectable label, and a third detectable label, respectively, wherein each detectable label is distinguishable from the other detectable labels, wherein each detectable label is encoded by a nucleic acid sequence linked to the respective gene, and wherein the expression of the nucleic acid sequence is dependent upon expression of the gene (co-expressed) in the respective cell population.

Embodiment 3: The non-human animal model of embodiment 2, wherein the detectable label is a fluorescent label or luminescent label.

Embodiment 4: The non-human animal model of embodiment 2, wherein each detectable label is a fluorescent label selected from the group consisting of green fluorescent protein (GFP), red fluorescent protein (RFP), and cyano fluorescent protein (CFP).

Embodiment 5: The non-human animal of any one of embodiments 1 to 4, wherein the animal model is a rodent.

Embodiment 6: The non-human animal of any one of embodiments 1 to 4, wherein the animal model is a mouse.

Embodiment 7: The non-human animal of any one of embodiments 1 to 4, wherein the animal model is a primate.

Embodiment 8: The non-human animal model of any one of embodiments 1 to 7, wherein the gene is expressed in B cells in humans.

Embodiment 9: The non-human animal model of any one of embodiments 1 to 8, wherein the gene is Lrba.

Embodiment 10: The non-human animal model of any preceding embodiment, wherein the non-human animal model comprises three populations of B cells, and wherein the three populations of B cells comprise:

(a) a first population of B cells having a wild-type lipopolysaccharide (LPS)-responsive beige-like anchor (Lrba) gene;

(b) a second population of B cells heterozygous for the Lrba gene; and

(c) a third population of B cells having an inactivated Lrba gene;

wherein the three populations of B cells include a transcription control cassette operably linked at the Lrba genomic locus.

Embodiment 11: A method for studying phenotypes, comprising:

providing a non-human animal model of any one of embodiments 1 to 10;

analyzing one or more of the phenotypes of the non-human animal model in the presence and/or absence of an exogenous agent.

Embodiment 12: The method of embodiment 11, wherein said analyzing comprises analyzing the characteristics and/or behavior of one or more of the cell populations of the animal model.

Embodiment 13. The method of claim 11 or 12, wherein said analyzing comprises subjecting cells of the animal model to flow cytometry.

Embodiment 14: The method of any preceding embodiment, wherein said analyzing comprises measuring the detectable label of one or more of the cell populations of the animals and, optionally, comparing the measured detectable label to that of one or both of the other detectable labels.

Embodiment 15: The method of any preceding embodiment, further comprising activing or deactivating the transcription control cassette to induce or inhibit expression of the gene.

Embodiment 16: The method of any preceding embodiment, wherein the exogenous agent is a small molecule or biologic molecule that is administered to the animal model.

Embodiment 17: A composition comprising a plurality of populations of cells from a single non-human animal, wherein said plurality of populations of cells comprise at least two of the following populations of cells:

(a) a first population of cells having a wild-type gene;

(b) a second population of cells heterozygous for the gene; and

(c) a third population of cells having an inactivated version of the gene;

wherein the three populations of cells include a transcription control cassette operably linked at the genomic locus.

Embodiment 18: The composition of embodiment 17, wherein the cell populations are B cell populations.

Embodiment 19: The composition of embodiment 18, wherein said plurality of populations of cells comprise at least two of the following populations of cells:

(a) a first population of B cells having a wild-type lipopolysaccharide (LPS)-responsive beige-like anchor (Lrba) gene;

(b) a second population of B cells heterozygous for the Lrba gene; and

(c) a third population of B cells having an inactivated Lrba gene;

wherein the three populations of B cells include a transcription control cassette operably linked at the Lrba genomic locus.

Embodiment 20: The composition of embodiment 18, wherein the three populations of cells comprise a first detectable label, a second detectable label, and a third detectable label, respectively, wherein each detectable label is distinguishable from the other detectable labels, wherein each detectable label is encoded by a nucleic acid sequence linked to the respective gene, and wherein the expression of the nucleic acid sequence is dependent upon expression of the gene (co-expressed) in the respective cell population.

Embodiment 21: The composition of any preceding embodiment, wherein the non-human animal is a rodent or primate.

Embodiment 22: The composition of any preceding embodiment, wherein the cells are B cells.

Embodiment 23: The composition of any preceding embodiment, wherein the composition comprises isolated B cells or a tissue comprising the B cells.

Embodiment 24: The composition of any one of embodiments 18 to 23, wherein the composition is peripheral blood from the non-human animal.

Embodiment 25: A method for studying phenotypes, comprising:
providing the composition of any one of embodiments 17 to 24;
analyzing one or more of the phenotypes of one or more of the cell populations in the presence and/or absence of an exogenous agent.

Embodiment 26: The method of embodiment 25, wherein said analyzing comprises analyzing the characteristics and/or behavior of one or more of the cell populations.

Embodiment 27: The method of embodiment 25 or 26, wherein said analyzing comprises subjecting cells of the animal model to flow cytometry.

Embodiment 28: The method of any one of embodiments 25 to 27, wherein said analyzing comprises measuring the detectable label of one or more of the cell populations of the animals and, optionally, comparing the measured detectable label to that of one or both of the other detectable labels.

Embodiment 29: The method of any one of embodiments 25 to 28, further comprising activing or deactivating the transcription control cassette to induce or inhibit expression of the gene.

Embodiment 30. The method of any one of embodiments 25 to 29, wherein the exogenous agent is a small molecule or biologic molecule that is administered to the animal model.

1. A non-human animal model comprising three populations of a cell type, wherein each cell population has a different genotype for a gene, wherein the three cell populations comprise:
(a) a first population of cells having a wild-type gene;
(b) a second population of cells heterozygous for the gene; and
(c) a third population of cells having an inactivated version of the gene;
wherein the three populations of cells include a transcription control cassette operably linked at the genomic locus of the gene.

2. The non-human animal model of claim 1, wherein the three populations of cells comprise a first detectable label, a second detectable label, and a third detectable label, respectively, wherein each detectable label is distinguishable from the other detectable labels, wherein each detectable label is encoded by a nucleic acid sequence linked to the respective gene, and wherein the expression of the nucleic acid sequence is dependent upon expression of the gene (co-expressed) in the respective cell population.

3. The non-human animal model of claim 2, wherein the detectable label is a fluorescent label or luminescent label.

4. The non-human animal model of claim 2, wherein each detectable label is a fluorescent label selected from the group consisting of green fluorescent protein (GFP), red fluorescent protein (RFP), and cyano fluorescent protein (CFP).

5. The non-human animal of claim 1, wherein the animal model is a rodent.

6. The non-human animal of claim 1, wherein the animal model is a mouse.

7. The non-human animal of claim 1, wherein the animal model is a primate.

8. The non-human animal model of claim 1, wherein the gene is expressed in B cells in humans.

9. The non-human animal model of claim 1, wherein the gene is Lrba.

10. The non-human animal model of claim 1, wherein the non-human animal model comprises three populations of B cells, and wherein the three populations of B cells comprise:
(a) a first population of B cells having a wild-type lipopolysaccharide (LPS)-responsive beige-like anchor (Lrba) gene;
(b) a second population of B cells heterozygous for the Lrba gene; and
(c) a third population of B cells having an inactivated Lrba gene;
wherein the three populations of B cells include a transcription control cassette operably linked at the Lrba genomic locus.

11. A method for studying phenotypes, comprising:
providing a non-human animal model of claim 1;
analyzing one or more of the phenotypes of the non-human animal model in the presence and/or absence of an exogenous agent.

12. The method of claim 11, wherein said analyzing comprises analyzing the characteristics and/or behavior of one or more of the cell populations of the animal model.

13. The method of claim 11, wherein said analyzing comprises subjecting cells of the animal model to flow cytometry.

14. The method of claim 11, wherein said analyzing comprises measuring the detectable label of one or more of the cell populations of the animals and, optionally, comparing the measured detectable label to that of one or both of the other detectable labels.

15. The method of claim 11, further comprising activing or deactivating the transcription control cassette to induce or inhibit expression of the gene.

16. The method of claim 11, wherein the exogenous agent is a small molecule or biologic molecule that is administered to the animal model.

17. A composition comprising a plurality of populations of cells from a single non-human animal, wherein said plurality of populations of cells comprise at least two of the following populations of cells:
(a) a first population of cells having a wild-type gene;
(b) a second population of cells heterozygous for the gene; and
(c) a third population of cells having an inactivated version of the gene;
wherein the three populations of cells include a transcription control cassette operably linked at the genomic locus.

18. The composition of claim 17, wherein the cell populations are B cell populations.

19. The composition of claim 17, wherein said plurality of populations of cells comprise at least two of the following populations of cells:
(a) a first population of B cells having a wild-type lipopolysaccharide (LPS)-responsive beige-like anchor (Lrba) gene;
(b) a second population of B cells heterozygous for the Lrba gene; and
(c) a third population of B cells having an inactivated Lrba gene;
wherein the three populations of B cells include a transcription control cassette operably linked at the Lrba genomic locus.

20. The composition of claim 17, wherein the three populations of cells comprise a first detectable label, a second detectable label, and a third detectable label, respectively, wherein each detectable label is distinguishable from the other detectable labels, wherein each detectable label is encoded by a nucleic acid sequence linked to the respective gene, and wherein the expression of the nucleic acid sequence is dependent upon expression of the gene (co-expressed) in the respective cell population.

21-30. (canceled)


SEQUENCE LISTING (6 sequences)

SEQ ID NO: 1 - DNA, 9919 bp, Homo sapiens
atctccgccgccacctcggtctcgctgctcctgggcggggggtgaggacgagtccggagtatctggggtttggcgttgttgtcagcctcggggagagagattggacaaatattctccaagaggaggagggcgacgccaaggactttccacatcaactgctttggggtatctccacaagttggaagagggaccctttcgttttgcattgcgtgtgttgtgctcattaccagtgcagcgactgccgtcccagggtgactctgagttgtcctttatcgtgagctagcaatggctagcgaagacaatcgtgtcccttccccgccaccaacaggtgatgacgggggaggtggagggagagaagaaacccctactgaagggggtgcattgtctctgaaaccagggctccccatcaggggcatcagaatgaaatttgccgtgttgaccggtttggttgaagttggagaagtatccaatagggatattgtagaaactgtctttaacctgttggtaggaggacagtttgatctggaaatgaatttcattatccaagaaggtgagagtattaactgcatggtggacctactggaaaaatgtgacattacgtgccaagcagaagtctggagcatgtttacagccattctgaagaaaagcatacggaatcttcaagtctgcactgaagtaggccttgttgaaaaagtgcttgggaaaattgaaaaagttgacaatatgatagcagatcttttggttgacatgttgggagtgctggctagctataatttgacagttcgcgagctaaagcttttcttcagtaaacttcaaggagataaaggacgatggcctccacatgctgggaagttgctgtctgtgttaaagcatatgcctcagaagtatggtcctgatgccttttttaactttccaggaaagagtgctgcagctattgcattacctcctatagccaaatggccataccagaatggttttacatttcatacatggcttagaatggatcctgtaaataacatcaatgtagataaggataaaccatatttgtattgtttcagaaccagcaaaggtcttggctattctgctcattttgttggaggctgtttgattgtaacatcaataaagtcaaaaggaaaaggctttcaacactgtgtgaaatttgatttcaagccacaaaagtggtatatggttaccatagtacacatctataaccgatggaagaatagtgaacttcgatgttatgtgaatggtgagctggcttcctatggagagataacatggtttgtcaacactagcgatacctttgacaaatgtttcctgggctcatcagaaacagcagatgctaatagagtattctgtggtcagatgactgcagtttaccttttcagtgaagctctaaatgcagctcagatatttgctatttatcagttgggcctgggatacaagggtacatttaaattcaaagcagaaagcgaccttttccttgctgagcatcacaaacttttattgtacgatgggaaactctctagtgccattgcattcacgtacaatccacgggctacagatgcccagctttgtcttgaatcatctcctaaggacaacccttcaatttttgttcattcaccacatgcactcatgctccaggatgtaaaggcagttttaacacattccatccaaagtgcaatgcattcaattggaggagtacaagtactatttccactttttgcacagttggattacaggcaatatttgtctgatgagattgatttgactatatgttcaaccttgctggcctttatcatggaattgttgaagaactcaattgctatgcaggaacagatgcttgcctgtaagggcttcttggtaataggatatagccttgaaaagtcttccaaatctcatgttagcagagcagtacttgaactttgccttgcattttcaaaatatctgagtaatctgcagaatgggatgcccctgctcaagcaattgtgtgatcacgttcttcttaatcctgccatatggattcataccccagccaaggttcaactgatgctctatacttatctgtccacggaattcattggtacagtcaacatatataacaccattcggagagttggaacagtgcttctcatcatgcacacgctgaagtactactactgggcagtgaatcctcaggatcgaagtggtatcaccccaaaaggattagatggaccgcgacctaatcaaaaagaaatgctttctctacgagcattcttgttgatgttcattaagcaattagtgatgaaggattctggagtaaaggaagatgaattacaggccattcttaattacctactgactatgcatgaggatgacaatctaatggatgtcctacagctgcttgttgcattaatgtcagaacaccctaactctatgattcctgcttttgaccaaaggaatgggttacgtgttatctacaaacttctggcatcgaaaagtgaaggaatcagggtacaagctcttaaggcaatgggttattttttaaaacatctggccccaaagaggaaagcagaagtcatgcttggacatggattgttttcattgctagctgaaaggctcatgcttcagacaaatttaatcacaatgaccacatataatgtgctgtttgagattcttatagaacagattggtactcaggtgatacataaacagcatccagatcctgattcttcagtgaagatacaaaaccctcagatactaaaagtaattgcgaccctacttcgaaattctccccagtgcccagagagcatggaggttcgcagagcctttctttctgacatgattaaactttttaataacagtagagaaaacaggaggagcttgctacaatgctctgtgtggcaagaatggatgctttctctctgctattttaatcctaagaattcagatgagcaaaagataacagaaatggtatacgccatattcagaatcctgctttaccatgcagtcaaatatgagtggggtggctggcgtgtatgggtagacactttatcaatcactcattcaaaggtcacttttgaaatacacaaagaaaaccttgccaatatatttagggaacagcaaggaaaagttgatgaagaaatagggctgtgttcttcaacttcagttcaagcagcctctggcattagaagggatattaatgtttcagtaggatcccagcaaccagatacgaaggattctcctgtctgtcctcatttcaccacaaatggtaatgaaaattcaagtatagagaagacaagttcactagaatctgcatctaatattgaactgcaaactactaatacatcttatgaagaaatgaaagctgagcaagaaaatcaggagttaccagatgaaggcactttggaagaaacactgacaaatgagacaaggaatgcagatgatttagaagtatcttctgacataatagaagctgtggctatttcctctaattcttttataacaactggcaaagattcaatgactgtcagtgaagtaactgcttctataagttctccttcagaagaggatgcctcagagatgccagaattcttggataaatctatagtagaggaagaggaagatgatgattatgtggaactgaaagtagaaggcagtcctactgaggaagctaatctacccacagagctccaagataacagtttgtctccagctgcatctgaagccggtgaaaaactggacatgtttggtaatgatgacaaattaatatttcaagaaggaaaacctgttactgaaaagcaaactgatactgaaactcaagattctaaagattctggaattcagactatgacagcatcagggtcttcagctatgtcaccagaaactactgtttcccaaatagctgtagaatcagaccttggtcagatgctggaggaagggaagaaagcaactaacctcactagagaaaccaaattaattaatgattgtcatggtagtgtctctgaggcttcttctgagcaaaagattgcgaagttggatgtttccaatgttgctacagatactgagaggctggagttgaaggccagtcccaacgtggaagcacctcaacctcatcgacatgtgcttgagatatcaaggcaacatgagcagccagggcaaggaatagcaccagatgcagttaatggacaaaggagggattccagatctactgtgtttcgtattcctgagttcaactggtctcagatgcatcaacgtttgctcactgatctattattttcaatagaaacagatatacagatgtggagaagccattcaacaaagacagttatggacttcgtgaatagcagtgataatgtcatctttgtacacaacacaattcatctcatctctcaagtgatggacaatatggtcatggcttgtgggggtatactgccattgctttcagctgctacatcggctacacatgaactggaaaatattgaacctactcaaggcctttcaatagaagcctctgtgacatttttgcagaggctaattagccttgtggatgtgcttatatttgcaagttctcttggctttactgaaattgaagctgaaaaaagtatgtcatctggaggaattttgcggcagtgtctccgactagtttgtgcagtcgcagtaaggaattgcttggagtgtcaacagcattcacaactgaaaactaggggagataaagccttgaaaccaatgcatagccttattcctttagggaaatctgcagcgaagagcccagtggacattgtgactggcggtatatctccagtaagagatcttgacaggcttctacaggacatggatattaatcggcttagggcagttgttttcagagacatagaggatagcaaacaagctcaatttttagccttggcagtagtatactttatctctgttcttatggtctccaagtacagagacattttggaaccccaaaatgaaaggcatagccagtcatgtacagaaactggcagtgaaaatgagaatgtatcactctctgaaatcacaccagcagcattcagcactttaactacggcatcagtggaagaatctgaaagcacatcatctgctcgaaggagggactcaggcattggggaagaaacagccactggtttaggaagccatgtggaagtaactcctcacacagcacctcctggtgtcagtgcaggcccagatgcaatcagcgaggtgctatctactctttctttagaagtcaataagtctccggaaaccaaaaatgatagaggaaatgacttggacactaaggctacaccgtcagtttcagtttcaaaaaacgtcaatgtgaaagacattctccgaagcttggttaacataccagcagatggagtcacagtggatcctgcccttctgccaccagcctgccttggagcccttggtgatctatctgtggaacaacccgtgcagttcagatcttttgacagaagtgtcattgttgcagcaaaaaagtcagcagtctcaccttccacctttaatacaagcatacctaccaatgctgtcagtgtggtttcctcagtagattcagcccaagcctcagatatgggaggagaatcaccaggcagtagatcatctaatgcaaaattgccctcagttccaacagttgattcagtttcacaagatccggtttcaaatatgagtattacagagaggcttgaacacgctttggaaaaggcagctcctctccttcgtgagatttttgtggattttgcaccttttctttctcggacacttttgggtagccatggacaagaactgcttatagaaggaacaagtctggtttgcatgaagtcgagtagttcagttgtggaattggttatgctactgtgttctcaggagtggcaaaattctattcagaagaatgcaggccttgcttttatcgaacttgtcaatgaaggaaggttgcttagccagacaatgaaggatcatctagtaagagtagcaaatgaagctgaatttatcctgagcaggcagagagcagaagatattcacagacatgcggaatttgagtcactgtgtgcccagtattctgcagacaaacgagaagatgagaagatgtgtgatcatttgataagagcagcaaaatatcgtgaccacgtgacagcaactcaactaatccagaaaattatcaacattctcacagacaagcatggagcctggggaaattctgcagtgagtcgtcctcttgagttctggcgccttgactactgggaagatgacttgcggcgccggcgacgatttgtgcgtaaccctctaggatcgacacatcctgaagcgacactaaaaacagccgtggaacatgccacagatgaagatatccttgctaaaggaaaacagtccatcaggagtcaggctttaggaaatcagaactcagaaaacgagatcctcctggaaggcgatgatgatactctgtcatccgtggatgagaaagatttagagaatcttgccggtcctgttagcctgagcacaccagctcagcttgtggccccctctgttgtagtaaagggcactctttctgtcacctcctccgaactctattttgaggtggatgaagaggatcctaacttcaaaaaaatcgaccccaagatcttggcatatacagaagggctgcatggaaaatggctgttcacagagatacgatcaatcttttctcgtcgttatcttttgcaaaatacagccctggagatctttatggcaaacagagttgctgtgatgttcaacttcccagaccctgcaacagtaaagaaagtggttaactatctacctcgtgttggcgttggaacaagttttggattgcctcaaaccagacgtatttcattagctagtccacgtcagctttttaaggcttctaatatgacccagcgatggcaacacagagagatatctaattttgagtacttgatgtttctcaacacgatagcaggacggagttataatgacttaaatcagtatccagtgtttccttgggtcatcactaattatgaatcagaagaactggatcttaccttgcccaccaacttcagagatttgtccaagccaataggagctctgaacccaaaaagagcagcattcttcgctgagcgttatgaatcatgggaagatgatcaagttccaaagtttcactatggtactcattactcaactgcaagttttgttcttgcatggctgctaagaatagaaccctttacaacttatttcctaaatttgcaaggaggcaaatttgatcatgcagatcgaactttttcatcaatttccagagcttggcgaaacagtcagcgtgatacctctgatattaaggagttgatccctgaattttattatctccctgagatgtttgtcaacttcaataattataatcttggagtgatggatgatgggacagtagtgtctgatgtcgaacttcctccttgggccaaaacctcagaagaatttgttcacataaacagattggccctggagagtgaatttgtttcctgccagcttcaccaatggattgatctcatttttggctataaacagcaaggaccagaagctgtccgagccctcaatgtgttctattacttgacctatgaaggagctgtcaatctgaattcaataactgatcctgtgttgagagaggctgttgaagctcaaatccgaagttttggacagactccttctcaactactcatagagccccatcctcccagaggttctgccatgcaagtgagtccattgatgttcacagacaaagcccagcaggatgttatcatggtcctcaagtttccctccaactcccctgttactcacgtggcagccaacacccagcctggtttggcaactcccgctgtgatcacagtcactgctaacaggttatttgcggtgaacaaatggcacaaccttcctgctcatcaaggtgctgtacaagaccagccataccagctgccagtggaaatcgatcctctcatagccagcaatacaggaatgcacaggaggcaaatcactgaccttttagaccaaagtattcaagtgcattcccagtgctttgtcatcacttcagacaaccgctatattctcgtctgtggcttctgggataaaagtttcagagtctattctacagacacaggaagattgatccaagtggtgtttggccattgggatgtcgtcacttgccttgctcgttctgagtcatatattgggggaaattgctacattctctcagggtcacgtgatgcaactcttttgctgtggtattggaatggaaaatgcagtgggattggagataacccaggtgagactgctgctcctcgggccattttgaccggccatgactatgaggtcacatgtgctgcggtgtgtgcggagctaggcctggtgttgagtggttcacaagaaggaccatgtctcatacattccatgaatggagacttgttgaggaccttggagggtcctgaaaactgcctgaaaccaaaactcattcaggcttcaagagagggtcattgtgtcatattctatgaaaacggcctcttctgtacattcagtgtgaatggaaaactccaggccacgatggaaacagatgataacataagagccatccagctgagccgagatgggcagtacctgctcacaggaggagacagaggagtggtcgtggtccggcaggtgtcggacctcaagcagctctttgcctatccaggatgtgacgctggaatccgggccatggcgctgtcttacgaccagaggtgcatcatttctggcatggcttcaggaagcattgtgctattttacaacgactttaaccggtggcatcatgaataccaaacccgctactgatggtgacagctgtacatcaactctgcccctaggatgagcagaagtacctggagcatcattctcttctaccacatctgaatgtaacttaaatttgctcaaagaagcaaaatattttttaaagttattaataattgctatttttgtagtctttgcttgacttttttgggggggattagcaaagcagaaaactggctgctgggttctgtagttttaaaaaatctatatttttagtcataatgagaagtctattttcaccaattatggaaacatacagtggagcaagtaaaccacttattccagctataatattatgaagaacatttcccatgcatctacaagcttgagaaataggattttcacagtggggaagtgggttacaatattggaataggaaaatttgatgctgtaatttgggtcctgtatttttccaaacaactgtgcttcttcagcactaatatttctgggatttaaatagtaatgtttaaattatggtaaatgtaatttaaaacatctcaattaagtctgtctatcaatattgttcttcaagcatgttttagacctagaaaagtatggtttgggggaggctattttattgtgtggttagcacaaggaatctaatttatagcaagtgtgaaatagttaccattttttcctgatctgtcatcttcatagcacaacaaaacgaaatgatggaaatgctcttgagctcacaacatttgtttttcttttaaagtaaatgcaagtaccaaagctcactactgcggtttgcctgtgcctggacaatgaggcggagccactgttgttttgttggggcacccccttccctccccgggtttgcaaatagaggctaccgggtgctgtattcagcaacacctgttttactatttgttattaaactatcatctccaccttccttttgattagcaatttgtactaagaaacgaaacaatgttatttggtggtgtataattctacttttctagtagattactgtgtggaattctgtgaaaaatatttgagaaaaggcctgtattgcataaataaattctttgtatgttgtgaaaaaaaaaaaaaaaaa

SEQ ID NO: 2 - DNA, 9900 bp, Mus musculus
atctccgccgccacctccgtctcgctgcgccccggcgggggtgaggacgaggccggagtctccgggctttggcgttgttgtcagcctccgggagagagattggacaaatagtctccaagaggaggagggcgacgaccaaggactttcctgcttttttgggtttctccgcaagttggaacagagaccctttttggtttagcgtttgcgtgtgtgttggtgcctcttctccgtgcagcgactgctggcccagggtgactctgacttgtcctttctcgtgaactagtcatggctagtgaagacaatcgtgccccttcccggccaccaacaggtgatgacgggggaggtggagggaaagaagaaacccctacggaagggggtgcgctgtctctgaagccggggctccccatcaggggcatcagaatgaaattcgccgtgctgacggggttggttgaagttggagaagtgtccaatagggatattgtagaaactgtctttaacctgctggtaggaggccagtttgatttggagatgaacttcattatccaggaaggtgagagcattatgtgcatggtggagctactggaaaagtgtgatgtcacttgccaagcagaagtctggagcatgtttacagccattctgaagaagagcatacgaaatcttcaagtctgcacagaagtaggcctcgtggaaaaagtacttgggaaaattgagaaagttgacagtatgattgcagatcttctcgttgacatgttgggagtgctggccagctataatttgacagttagagaactaaagcttttcttcagtaaacttcaaggagataaaggacaatggcctcctcacgctgggaagctgctgtcagtgttaaagcacatgcctcagaagtacggtcctgacgcctttttcaactttccaggaaagagcgctgcagctattgcattacctcctatagccagatggccttaccagaatggttttacatttcacacctggcttagaatggatcctgtaaataatattaatgttgacaaggataaaccatatttgtattgtttcagaaccagcaaaggtcttggctattctgctcattttgttggaggctgtttgattataacctcaataaagtcaaaaggaaaaggcttccaacattgtgtgaaatttgatttcaagccacaaaagtggtacatggtaactatagtacacatttataaccggtggaagaacagtgagcttcgctgttacgtgaatggggaacttgcttcctatggagagataacatggtttgtcaacaccagtgatacctttgacaaatgcttcctgggatcatcggagacagcggatgcgaatcgggtgttctgtggccagatgactgcggtttacctgttcagtgatgctcttaatgcagctcagatttttgccatttatcagttgggcctggggtacaagggcacatttaaatttaaagcagaaagtgaccttttcctcgctgagcaccataaactgttactgtatgacggcaagctgtccagtgccattgccttcacgtacaacccgcgggccacagatgcccagctgtgcctggagtcgtctcccaaggacaacccctccatcttcgtccactcgccacacgcactcatgctccaggatgtaaaggcagttttgacacattccatccagagcgccatgcactccataggaggagtacaagttctgtttccgctcttcgcccagctggactacaagcaatatttatctgatgaggttgacttgactatctgtacaaccttgctggcctttatcatggagttgctgaagaactcaattgctatgcaggagcagatgcttgcttgtaagggctttttggtaataggttatagccttgaaaagtcttccaaatcccacgtcagcagagcagtacttgaactttgccttgcgttttcaaagtatttgagcaatctgcagaatgggatgcccctcctgaaacagctgtgtgatcacattcttcttaaccctgctgtatggatccacactccagccaaggttcagttgatgctttatacttatctatccactgagttcattgggacggtcaacatctataacactattcggagagttggaacagtgctgctcatcatgcacacgctcaagtactactactgggcagtgaatcctcaggaccgaagtggtatcacccccaaaggattagatggaccaagacccaatcagaaagaaatactttctctgcgagcattcttactgatgttcattaagcagttagtgatgaaggactctggagtgaaggaagatgagctacaggccattcttaactacctgctgactatgcatgaggacgacaatctaatggatgtcctacagctgcttgttgcgttgatggcggagcatcctaactccatgattccggcttttgaccaaaggaatgggctgcgtgttatctataaacttctggcatcgaagagcgaaggaatccgagtgcaggcactcaaggcactgggatactttttaaagcatctggcgccaaagagaaaagctgaagtcatgcttgggcatggattgttctcgctgctagctgagagacttatgcttcagacaaatctaatcacaatgaccatgtataatgttctgtttgagattcttatagaacagatctgtactcaggtgatccacaaacagcatccagatcccgattctacagtaaaaatacaaaaccctcagatactaaaagtaattgcgaccctacttcgaaattctccccagtgcccagagagcatggaggttcgccgagcatttctttctgacatgattaaactttttaataacagtagagaaaacaggaggagcttgctgcagtgctctgtgtggcaagaatggatgctttctctctgctattttaatcctaagaattcagatgagcaaaagataacagagatggtgtacgccatattcagaattctgctttaccatgcagtcaaatacgagtggggtggctggcgagtgtgggtagacaccttatccatcacacattcaaaggttacttttgaaatacacaaagaaaaccttgccaatatatttagggaagagcagcggaaaggtgatgaagaaacaggaccgtgttcttcaagcctggttccagaaggcactggtgctactaggggtgttgatgtttcagtggggtcccagcacgaagatcggaaggattctcccatctctcctcatttcactagaaatagtgatgagaactcaagtattgggagggcaagttcaatagattctgcatccaacactgaactgcagacacatgatatgtctagtgatgaaaagaaagtagagcgagaaaatcaagagttactggatcaagccaccgtggaagaaacagctacaaatggggcaaaagatgacttagaaacgtcttctgatgcagcagagccagtgactattaactctaattctttggaaccaggcaaagatacagtgaccatcagtgaagtaagtgcttccataagttctccctcagaagaagatgctgcagagatgccagaattactggaaaagtctggagtagaggaagaagaggatgatgactatgtggaactgaaggtagaaggcagccctactgaggaagctggtctgcccacagagctccaaggtgagggtttgtctgtagctgcctctgaaggcagagaagaaccagatatgtgtggtcatggctgtgaagttcaagtggaagcacctattactaaaattcataatgatcctgaaactacagattctgaggattctaggtttccaactgtggccacagcagggtctttagctacctcatcagaagttcctgttccccaggcaactgtacagtcagacagccatgagatgctggatggagggatgaaagcaactaaccttgccggagaaactgagtcagttagtgattgtgctgataatgtctctgaggctcctgccacttctgagcagaagattaccaaactggatgtttctagtgtggcttcagatactgagaggtttgaattgaaggccagtacgagcacagaagcaccacagcctcaacgacatgggcttgagatatcaaggcaacaggagcagacagcacaaggaacagcaccagatgcagtagaccaacaaaggagggactccagatccaccatgtttcgcattcctgagttcaagtggtctcagatgcatcaacgtctgctcactgatctcttattttccatagaaacagatatacagatgtggagaagccattcaacaaagacagttatggacttcgtgaatagcagtgataatgtcatctttgtgcacaacacaattcatctcatctctcaagtgatggacaatatggtcatggcttgtgggggtatactgccattactctcagctgctacctcggccacacatgagctggagaacattgagcctactcaaggcctttcaatagaagcctcagtgacatttcttcagaggctaattagccttgtggatgtgcttatatttgcaagttctcttggttttactgaaattgaagctgaaaaaaatatgtcatctggaggaattctgcgacagtgtctccgattggtgtgtgcagtagctgtaaggaattgcttagagtgtcagcaacattcacagctgaaagccagaggagatacagcaaagagctcgaaaacaatacatagcctgattcccatggggaaatctgcagcaaagagcccagtagacattgtgactggtggtatatctccggtaagagatcttgacaggctcctgcaagacatggacattaatcggcttcgagcagttgttttcagagacatcgaggacagcaagcaagctcaattcttggctttggctgttgtgtactttatctctgttctgatggtctcaaagtacagagacattttggaaccccaagatgaaagacacagtcagtctcttaaggaaactagtagtgataatgggaatgcatcacttcctgatgcagaaaacacaccagcagaatttagttctttaactctgtcgtcggtggaagaatcattggaaggcacctcatgcactcgcaggagggactcaggccttggtgaggaaacagcctctggcttaggaagcggtctgtctgtcgcttctccagcggcgcctctgggtgtcagcgcggggccagatgccatcagcgaggtgctgtgcactctttctttggaagtcaataaatctcaggaaacaagaattgatggaggaaacgagttggacaggaaggtgacaccatcagtcccagtttcaaaaaatgtcaacgtgaaggacattctccggagcttggtgaatatgccagcagatggagtcacagtggatccagcccttctgcccccagcctgccttggtgctcttggagatctgtctgttgacccacccatgcagttcagatcctttgacagaagtgtcatcattgcaacaaagaaatcatcagtcttaccttccgcccttactacaagtgcacctagtagcgccgtcagtgtggtgtcttcagtagatcccacccacgcctcagacaccggaggagagtcaccaggaagtagatctcctaatgcaaaactgccctcagttgcagcagttggctccgtcccacaagacccagctgcacacatgagtattacagaaaggctagagcatgcactggagaaggcagctcctctgctccgagagatttttgtggattttgcacctttcctttctcggacacttttaggtagtcatggacaagaactgctgatagaaggaacaagcctggtctgcatgaagtccagcagttcagttgtggagctggtcatgctgctgtgttcccaggagtggcagaattctattcagaagaatgcaggccttgcttttattgaacttgtcaatgaaggaaggttacttagccaaacaatgaaggatcatttagtaagagtagcaaatgaagccgaatttatcctgagcaggcagagagcagaggatattcacagacatgcagaatttgagtcattgtgtgctcaatactctgcagacaaaagagaagaggagaagatgtgtgatcatttgataagagcagctaaatatcgagaccatgtgaccgcaactcagttaatccagaaaattatcaacctcctcacagacaagcatggagcctgggggagctctgcagtaagtcgtcctcgtgagttctggcgccttgactactgggaagatgacttgaggcgccggcgacgatttgtgcgtaaccctctaggatcgacacatcctgaagcgacactaaaaacagccgtggaacatgctgcagatgaagacatccttgctaaaggaaaacagtccatcaagagtcaggctttaggaaatcagaactctgaaaacgaggccctcctggaaggcgacgacgatactctgtcatccgtggatgagaaagatttagagaatcttgccggtcctgttagcctgagcaccccagctcagcttgtggccccctctgttgtagtaaaaggcactctctctgtcacttcctctgaactctattttgaggtggatgaagaggatcccaacttcaagaaaattgacccaaagatcttggcatatacagaaggtctgcatggaaaatggctgttcacagagataagatcaatcttttctcgccgttatcttttgcaaaatacagctctggagatctttatggcaaacagagttgctgtaatgttcaacttcccagaccctgccacagtaaagaaagtggtgaactatctacctcgtgttggtgtgggaaccagttttggattacctcagaccaggcgtatttcattagccactccacgtcagctattcaaagcttcaaatatgactcagcggtggcagcacagagagatttcaaacttcgagtacttgatgtttctcaacacaatagcagggcggagttataatgacctaaatcagtatcctgtgtttccctgggtcatcactaattatgagtcagaagaattagatcttaccttgccaagcaatttcagagatttgtccaagccaataggagctttgaatcccaaacgagcagcattctttgcagagcgatttgagtcatgggaagatgatcaagttccaaagttccactatggtactcattactcaactgcaagttttgttcttgcgtggcttctaagaatagaaccttttacaacttactttctaaatttacaaggagggaaatttgatcatgcagataggacgttctcctcagtctccagagcatggcgaaacagtcagcgcgacacatctgacattaaggaattgattcctgaattttattatctccctgagatgtttgtcaacttcaataattataaccttggagtgatggatgatgggacagtggtgtctgatgttgaacttcctccttgggccaaaacctcggaagaattcgttcgcataaacagactggccctggagagtgaatttgtttcctgccagcttcaccaatggattgatcttatttttggctataaacaacaaggaccagaggcagtgcgagccctcaatgtgttctattacctaacctatgaaggagctgtcaacctgaactcaataactgatcctgtgctgagagaggctgtggaagctcagatccgaagttttggacagacgccttcgcaactgctcatagagccccaccctccccgaggttcggccatgcaggcgagtccattgatgttcacagaccaagcccagcaagatgtcatcatggtcctaaagttcccttcgaattctccagtcacccacgttgcggccaacacccagccaggcctggcaatgcctgctgtcatcactgtcactgctaacaggctctttgccgtgaacaagtggcacaaccttcctgctcaccaaggtgctgtacaagaccagccataccagctgccagtggaaatcgatcctctcatagcctgcggcacagggacacacaggaggcaggtaacagacctcctggaccagagcatccaagtgcactcccagtgcttcgtcatcacttccgacaaccgctacattctcgtctgtggcttctgggataagagtttccgggtctactccacagatacaggaaaattgatccaagtggtgtttggccattgggatgttgtcacttgcctagctcgctctgagtcgtacatagggggaaattgttacatcctctcggggtcacgtgacgcaactcttcttctgtggtactggaatgggaagagcagcgggattggagataaccctggcggtgaaactgccacccctcgggccattctcacaggccatgactacgagatcacctgtgctgctgtctgcgctgagctgggcctggtgttaagtggttcccaagaagggccgtgtctcatacactccatgaatggagacctgttaaggactttggagggtcccgaaaattgcctgaaaccaaaactcatccaagcatcgagagagggtcactgtgtcatcttctatgaaaacggctgcttctgcaccttcagtgtgaacgggaagctgcaggccaccgtggagacggatgatcacataagggccatacagctgagcagagatgggcagtacctgctcacaggaggagacaacggggtggtgatagtgcggcaggtgtctgacctcaagcagctctttgcctacccaggctgtgatgctggaatccgggccatggccctttcttttgaccagaggtgcatcatttctggaatggcttcgggaagcattgtcctgttttacaatgactttaaccgctggcatcatgagtaccagacccgctactgaggggaacgaatgcaccgtggtcctgtttcagcccaagcaaggaaggacttcaaacaccactctatagaaatagctatcacatctgaatgtaacttaaatttgcttgaacaagcaaaatattttttaaagttaattgctatttttgtagactttgctcgacattttggggggggggggggtgggaacagcacaagcagggaactggctgctgggttctgtagtttacaaaatctatatttttagtcctaatgaaaagtctattttcaccaattatggatacagacagtggaaccaataatcccactaattcttgctacatgtgaaaaaccatttcccacttatctgtaatcttaagaaatatgattttagcaatagggaaatgggttcaaatgggggacagggaaatttattctgtactgggtcctgtatttttctagacaattgtgcttcttcagtgctgacatttctagggttttaatagtaatgtttaaattatgacgtgtaatttaaaacatctcagtgaattatttctgtattattccccttcaagcatgtgttagacctagaaaattatggtttagggaggctactttattttactgtgtgttaaacagcaaagatctaagtttatcaatcataaatactgcttttcctgatctgccgtcctcatggcagagagtggaatgatgggaccgctcttgggctcgcaaccttttaagcactacagcctgcccgaggccaccagtgccctgctaagccagagccaggtgggccactccccatgtccactggcccacagtcctgtctcaagatcagaggcgaccaggtactgtgtaagcagcacccaccttacccctatttgttatgaagctatctttaccttccttttgactagtaatttgtatcaagaaaatgttattttggtgtcatataattctactttttctagtagattgctttatggaattctgtgaaaatatttgagcagaggtctgtattacataaataaattctttgtatgttgtgaacttgaaaaaaaaaaaaaaaaaa

SEQ ID NO: 3 - DNA, 70 bp, Artificial
  Lrba/SD/loxP portion of Lrba BAC subclone plasmid
tttctcgtgaacgcgtcttcagggtgagtttataacttcgtataatgtatgctatacgaagttatacgcg

SEQ ID NO: 4 - DNA, 71 bp, Artificial
  STOP/loxP portion of Lrba BAC subclone plasmid
ttgacattgattaatacgactcactatagggctcgagataacttcgtataatgtattacaaaggcagcggc

SEQ ID NO: 5 - DNA, 71 bp, Artificial
  P2A/Lrba portion of Lrba BAC subclone plasmid
ccacaaacttttcactcctgaaacaggccggggatgtagaggagaaccccggccctatggctagtgaagac

SEQ ID NO: 6 - DNA, 30 bp, Artificial
  transcription control cassette
cttgtcctttctcgtgaactagtcatggct